(19) United States
(12) Patent Application Publication
(10) Pub. No.: US 2025/0208014 A1
(43) Pub. Date: Jun. 26, 2025

(54) METHODS AND SYSTEMS RELATING TO QUALITY CONTROL OF CONSTRUCTION MATERIALS

(71) Applicant: GIATEC SCIENTIFIC INC., NEPEAN (CA)

(72) Inventors: ANDREW FAHIM, OTTAWA (CA); POURIA GHODS, GLOUCESTER (CA); ROUHOLLAH ALIZADEH, NEPEAN (CA); MUSTAFA SALEHI, NEPEAN (CA)

(21) Appl. No.: 18/846,973

(22) PCT Filed: Mar. 14, 2023
(86) PCT No.: PCT/CA2023/050327

§ 371 Date: Sep. 13, 2024

Related U.S. Application Data
(60) Provisional application No. 63/319,581, filed on Mar. 14, 2022.

(51) Int. Cl. G01N 11/00; G01N 3/24; G01N 33/38

(52) U.S. Cl. CPC G01N 11/00; G01N 3/24; G01N 33/383; G01N 2011/0033; G01N 2011/0093; G01N 2203/0025; G01N 2203/021; G01N 2203/0676; G01N 2203/0694

(57) ABSTRACT

With increasing demands for cost reductions, profitability, tighter construction deadlines and potential liabilities construction companies, raw material suppliers, infrastructure owners, etc. are seeking cost effective systems, method and processes relating to the quality control of said construction materials. Accordingly processes, systems and methods are disclosed relating to concrete and other construction materials such as automatic slump measurement, automatic load measurement, artificial intelligence—machine learning optimization of material mixes, and automatic ingestion of data from unstructured documents to provide data to artificial intelligence—machine learning processes.

### CROSS-REFERENCE TO RELATED APPLICATIONS

This patent application claims the benefit of priority to World Intellectual Property Organization Application PCT/CA2023/050327 filed Mar. 14, 2023; which itself claims the benefit of priority to U.S. Provisional Patent Application 63/319,581 filed Mar. 14, 2022; the entire contents of each being incorporated herein by reference.

### FIELD OF THE INVENTION

This patent application relates to construction materials and more particularly to the systems, method and processes relating to the quality control of said construction materials.

### BACKGROUND OF THE INVENTION

Globally, the construction industry output is forecast to rise from US$10.8 trillion in 2017 to US$12.9 trillion in 2022. A wide variety of materials are employed within the construction industry of which some are chemically active materials, e.g., concrete, that often need to be analyzed so as to determine the structural properties parameters, particularly strength and other physical-mechanical properties of the final cured product, such as its potential for shrinkage. With increasing demands for cost reductions, profitability, tighter construction deadlines and potential liabilities construction companies, raw material suppliers, infrastructure owners, etc. are seeking cost effective systems, method and processes relating to the quality control of said construction materials.

Other aspects and features of the present invention will become apparent to those ordinarily skilled in the art upon review of the following description of specific embodiments of the invention in conjunction with the accompanying figures.

### SUMMARY OF THE INVENTION

It is an object of the present invention to mitigate limitations within the prior art relating to construction materials and more particularly to the systems, method and processes relating to the quality control of said construction materials.

In accordance with an embodiment of the invention there is provided a method comprising:
providing a drum;
providing at least one of an accelerometer and a gyroscope attached to the drum;
determining a torque of an engine rotating the drum at a plurality of rates of revolution;
determining in dependence upon the torque of the engine at the plurality of different revolutions per minute (RPM), parameters of the drum, and a weight of a construction material within
the drum a yield stress of the construction material; wherein the yield stress of the construction material is established in extrapolating the torque of the engine at the plurality of rates of revolution to a torque required at zero (0) RPM.

In accordance with an embodiment of the invention there is provided a method comprising
providing a drum for containing a load of concrete;
providing a hydraulic pressure sensor measuring pressure of a hydraulic system associated with the drum;
providing a sensor to monitor an angular velocity of the drum;
monitoring the hydraulic pressure of the hydraulic system over a range of angular velocities with the drum empty;
monitoring the hydraulic pressure of the hydraulic system over a range of angular velocities with a load of concrete within the drum;
determining in dependence upon the difference in hydraulic pressures between the drum empty and loaded with concrete over the range of angular velocities and the range of angular velocities a shear rate and a shear stress applied to the concrete;
determining a yield stress of the concrete from the shear rate and a shear stress; and
determining in dependence upon the yield stress of the concrete a slump of the concrete.

In accordance with an embodiment of the invention there is provided a method comprising
providing a drum for containing a load of concrete;
providing a hydraulic pressure sensor measuring pressure of a hydraulic system associated with the drum;
providing a sensor to monitor an angular velocity of the drum;
monitoring the hydraulic pressure of the hydraulic system over a range of angular velocities with the drum empty;
monitoring the hydraulic pressure of the hydraulic system over a range of angular velocities with a load of concrete within the drum;
determining in dependence upon the hydraulic pressures over the range of angular velocities when the drum is empty and the range of angular velocities a system efficiency value; and
determining in dependence upon the hydraulic pressures over the range of angular velocities when the drum is loaded, the range of angular velocities and the system efficiency value a size of the concrete load.

In accordance with an embodiment of the invention there is provided a method comprising:
establishing a first pressure measurement from a first diaphragm based pressure sensor in contact with a column of a construction material at a first position with respect to the column of the construction material;
establishing a second pressure measurement from a second diaphragm based pressure sensor in contact with a column of a construction material at a second position with respect to the column of the construction material;
establishing a pressure differential in dependence upon the first pressure measurement and the second pressure measurement;
establishing a vertical separation between the first diaphragm based pressure sensor and the second diaphragm based pressure sensor; and
establishing at least one of a flowability measurement or slump measurement of the construction material.

In accordance with an embodiment of the invention there is provided a method comprising:
establishing a model comprising a formwork and a body of liquid concrete poured into the formwork;
executing a simulation process upon the model over a period of time to establish a measure of temperature increase of the body of liquid concrete at a plurality of points and a plurality of time; wherein
the simulation process has been correlated to a plurality of physical structures which have been modelled with the simulation process.

In accordance with an embodiment of the invention there is provided a method comprising:
establishing an initial set of documents;
labelling each document of the set of documents to identify within that document of the set of documents a plurality of regions within the document of the set of documents containing content of interest;
establishing a training set of documents from the labelled set of documents;
employing at least one of a machine learning (ML) based rule generator and an artificial intelligence (AI) based rule generator upon the training set of documents to generate a set of rules for extracting content in dependence upon the labelled content in each document of the training set of documents; and
storing the rules within the database.

In accordance with an embodiment of the invention there is provided a method comprising
performing an electrical impedance measurement of at least one of a cement and a mortar with a sensor embedded within performing a permittivity measurement of the at least one of a cement and a mortar with the sensor;
determining in dependence upon the electrical impedance measurement and the permittivity measurement at least one of a porosity of the cement, a time of set of the at least one of the cement paste and the mortar and a compressive strength of the at least one of the cement and the mortar.

In accordance with an embodiment of the invention there is provided a method comprising:
establishing a model in dependence upon applying watershed transforms to images of particulates of varying sizes to perform particle segmentation;
employing a machine learning process or artificial intelligence based process to classify images of a particulate mixture to identify a plurality of aggregate particles within the particulate mixture by isolating the plurality of aggregate particles from artifacts within acquired images of the particulate mixture caused by at least one of image noise and distortion;
establishing one or more parameters of the plurality of aggregate particles in dependence of the model.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 depicts an exemplary network environment within which embedded sensors can be employed according to and supporting embodiments of the invention may be deployed and operate; and

FIG. 2 depicts an exemplary electronic device supporting communications both to a network such as depicted in FIG. 1 and with embedded sensors according to and supporting embodiments of the invention;

FIG. 3 depicts an embedded sensor methodology for data logging concrete properties from initial mix through pouring, curing, and subsequently according to an embodiment of the invention;

FIG. 4 depicts a sensor according to the embodiment of the invention prior to deployment, in deployed state and being read in deployed state; and

FIG. 5 depicts exemplary embeddable sensors according to embodiments of the invention;

FIG. 6 depicts an exemplary process flow for the verification and/or specification of a construction material manufacturing composition based upon acquired material performance data from previous deployments acquired using sensors according to embodiments of the invention;

FIG. 7A depicts an exemplary process flow for optimizing a manufacturing specification for a construction material according to an embodiment of the invention exploiting machine learning and artificial intelligence;

FIGS. 7B and 7C depict exemplary schematics of extended sensor integration within a concrete production facility in conjunction with embedded sensors monitoring one or more portions of a subsequent life cycle of the construction material for optimizing a mixture for a construction material according to an embodiment of the material;

FIG. 8 depicts an exemplary process flow for optimizing a construction material during transportation according to an embodiment of the invention exploiting machine learning and artificial intelligence;

FIG. 9 depicts an exemplary process flow for optimizing a construction material for reduced cost/waste based upon embedded sensor data exploiting machine learning and artificial intelligence;

FIG. 10 depicts an exemplary process flow for service life assessment for an infrastructure element exploiting monitored installations of its construction material according to an embodiment of the invention exploiting machine learning and artificial intelligence;

FIG. 11A depicts a sensor configuration for a drum of a concrete delivery truck employed as part of a system for determining in situ yield stress measurements of a construction material according to an embodiment of the invention;

FIG. 11B depicts a pressure sensor and data acquisition for a concrete delivery truck employed as part of a system for determining in situ yield stress measurements of a construction material according to an embodiment of the invention;

FIG. 12 depicts readings from a sensor as depicted in FIG. 11 under steady state and varying rotation rates of the drum according to an embodiment of the invention;

FIG. 13 depicts hydrostatic pressure measurements obtained with pair of diaphragm based sensors versus vertical sensor separation for two different concrete mixes wherein the pressure differential is employed to establish a slump measurement of the concrete according to an embodiment of the invention;

FIG. 14 depicts temperature versus time for simulations of two different concrete mixes according to a model according to an embodiment of the invention;

FIG. 15 depicts schematically a software application according to an embodiment of the invention;

FIG. 16 depicts a process flow for a process for defining data to be extracted from unstructured documents according to an embodiment of the invention; and

FIGS. 17 to 19 depict exemplary unstructured documents labelled with regions of content to be extracted where the labelled unstructured documents are employed according to an embodiment of the invention.

### DETAILED DESCRIPTION

The present invention is directed to construction materials and more particularly to the systems, method and processes relating to the quality control of said construction materials.

The ensuing description provides representative embodiment(s) only, and is not intended to limit the scope, applicability, or configuration of the disclosure. Rather, the ensuing description of the embodiment(s) will provide those skilled in the art with an enabling description for implementing an embodiment or embodiments of the invention. It being understood that various changes can be made in the function and arrangement of elements without departing from the spirit and scope as set forth in the appended claims. Accordingly, an embodiment is an example or implementation of the inventions and not the sole implementation. Various appearances of “one embodiment,” “an embodiment” or “some embodiments” do not necessarily all refer to the same embodiments. Although various features of the invention may be described in the context of a single embodiment, the features may also be provided separately or in any suitable combination. Conversely, although the invention may be described herein in the context of separate embodiments for clarity, the invention can also be implemented in a single embodiment or any combination of embodiments.

Reference in the specification to “one embodiment,” “an embodiment,” “some embodiments” or “other embodiments” means that a particular feature, structure, or characteristic described in connection with the embodiments is included in at least one embodiment, but not necessarily all embodiments, of the inventions. The phraseology and terminology employed herein is not to be construed as limiting but is for descriptive purpose only. It is to be understood that where the claims or specification refer to “a” or “an” element, such reference is not to be construed as there being only one of that element. It is to be understood that where the specification states that a component feature, structure, or characteristic “may,” “might,” “can” or “could” be included, that particular component, feature, structure, or characteristic is not required to be included.

Reference to terms such as “left,” “right,” “top,” “bottom,” “front” and “back” are intended for use in respect to the orientation of the particular feature, structure, or element within the figures depicting embodiments of the invention. It would be evident that such directional terminology with respect to the actual use of a device has no specific meaning as the device can be employed in a multiplicity of orientations by the user or users.

Reference to terms “including,” “comprising,” “consisting,” and grammatical variants thereof do not preclude the addition of one or more components, features, steps, integers, or groups thereof and that the terms are not to be construed as specifying components, features, steps, or integers. Likewise, the phrase “consisting essentially of,” and grammatical variants thereof, when used herein is not to be construed as excluding additional components, steps, features integers or groups thereof but that the additional features, integers, steps, components, or groups thereof do not materially alter the basic and novel characteristics of the claimed composition, device, or method. If the specification or claims refer to “an additional” element, that does not preclude there being more than one of the additional element.

A “wireless standard” as used herein and throughout this disclosure, refer to, but is not limited to, a standard for transmitting signals and/or data through electromagnetic radiation which may be optical, radiofrequency (RF) or microwave although typically RF wireless systems and techniques dominate. A wireless standard may be defined globally, nationally, or specific to an equipment manufacturer or set of equipment manufacturers. Dominant wireless standards at present include, but are not limited to IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1000, Bluetooth, Wi-Fi, Ultra-Wideband and WiMAX. Some standards may be a conglomeration of sub-standards such as IEEE 802.11 which may refer to, but is not limited to, IEEE 802.1a, IEEE 802.11b, IEEE 802.11g, or IEEE 802.11n as well as others under the IEEE 802.11 umbrella.

A “wired standard” as used herein and throughout this disclosure, generally refer to, but is not limited to, a standard for transmitting signals and/or data through an electrical cable discretely or in combination with another signal. Such wired standards may include, but are not limited to, digital subscriber loop (DSL), Dial-Up (exploiting the public switched telephone network (PSTN) to establish a connection to an Internet service provider (ISP)), Data Over Cable Service Interface Specification (DOCSIS), Ethernet, Gigabit home networking (G.hn), Integrated Services Digital Network (ISDN), Multimedia over Coax Alliance (MoCA), and Power Line Communication (PLC, wherein data is overlaid to AC/DC power supply). In some embodiments a “wired standard” may refer to, but is not limited to, exploiting an optical cable and optical interfaces such as within Passive Optical Networks (PONs) for example.

A “user” as used herein may refer to, but is not limited to, an individual or group of individuals. This includes, private individuals, employees of organizations and/or enterprises, members of community organizations, members of charity organizations, men, women, and children. In its broadest sense the user may further include, but not be limited to, mechanical systems, robotic systems, android systems, etc. that may be characterised by an ability to exploit one or more embodiments of the invention.

A “sensor” as used herein may refer to, but is not limited to, a transducer providing an electrical output generated in dependence upon a magnitude of a measure and selected from the group comprising, but is not limited to, environmental sensors, medical sensors, biological sensors, chemical sensors, ambient environment sensors, position sensors, motion sensors, thermal sensors, infrared sensors, visible sensors, RFID sensors, and medical testing and diagnosis devices.

A “portable electronic device” (PED) as used herein and throughout this disclosure, refers to a wireless device used for communications and other applications that requires a battery or other independent form of energy for power. This includes devices, but is not limited to, such as a cellular telephone, smartphone, personal digital assistant (PDA), portable computer, pager, portable multimedia player, portable gaming console, laptop computer, tablet computer, a wearable device, and an electronic reader.

A “fixed electronic device” (FED) as used herein and throughout this disclosure, refers to a wireless and/or wired device used for communications and other applications that requires connection to a fixed interface to obtain power. This includes, but is not limited to, a laptop computer, a personal computer, a computer server, a kiosk, a gaming console, a digital set-top box, an analog set-top box, an Internet enabled appliance, an Internet enabled television, and a multimedia player.

A “server” as used herein, and throughout this disclosure, refers to one or more physical computers co-located and/or geographically distributed running one or more services as a host to users of other computers, PEDs, FEDs, etc. to serve the client needs of these other users. This includes, but is not limited to, a database server, file server, mail server, print server, web server, gaming server, or virtual environment server.

An “application” (commonly referred to as an “app”) as used herein may refer to, but is not limited to, a “software application,” an element of a “software suite,” a computer program designed to allow an individual to perform an activity, a computer program designed to allow an electronic device to perform an activity, and a computer program designed to communicate with local and/or remote electronic devices. An application thus differs from an operating system (which runs a computer), a utility (which performs maintenance or general-purpose chores), and a programming tools (with which computer programs are created). Generally, within the following description with respect to embodiments of the invention an application is generally presented in respect of software permanently and/or temporarily installed upon a PED and/or FED.

An “enterprise” as used herein may refer to, but is not limited to, a provider of a service and/or a product to a user, customer, or consumer. This includes, but is not limited to, a retail outlet, a store, a market, an online marketplace, a manufacturer, an online retailer, a charity, a utility, and a service provider. Such enterprises may be directly owned and controlled by a company or may be owned and operated by a franchisee under the direction and management of a franchiser.

A “service provider” as used herein may refer to, but is not limited to, a third-party provider of a service and/or a product to an enterprise and/or individual and/or group of individuals and/or a device comprising a microprocessor. This includes, but is not limited to, a retail outlet, a store, a market, an online marketplace, a manufacturer, an online retailer, a utility, an own brand provider, and a service provider wherein the service and/or product is at least one of marketed, sold, offered, and distributed by the enterprise solely or in addition to the service provider.

A “third party” or “third party provider” as used herein may refer to, but is not limited to, a so-called “arm's length” provider of a service and/or a product to an enterprise and/or individual and/or group of individuals and/or a device comprising a microprocessor wherein the consumer and/or customer engages the third party but the actual service and/or product that they are interested in and/or purchase and/or receive is provided through an enterprise and/or service provider.

A “user” as used herein may refer to, but is not limited to, an individual or group of individuals. This includes, but is not limited to, private individuals, employees of organizations and/or enterprises, members of community organizations, members of charity organizations, men, and women. In its broadest sense the user may further include, but not be limited to, software systems, mechanical systems, robotic systems, android systems, etc. that may be characterised by an ability to exploit one or more embodiments of the invention. A user may also be associated through one or more accounts and/or profiles with one or more of a service provider, third party provider, enterprise, social network, social media etc. via a dashboard, web service, website, software plug-in, software application, and graphical user interface.

A “wearable device” or “wearable sensor” relates to miniature electronic devices that are worn by the user including those under, within, with or on top of clothing and are part of a broader general class of wearable technology which includes “wearable computers” which in contrast are directed to general or special purpose information technologies and media development. Such wearable devices and/or wearable sensors may include, but not be limited to, smartphones, smart watches, e-textiles, smart shirts, activity trackers, smart glasses, environmental sensors, medical sensors, biological sensors, physiological sensors, chemical sensors, ambient environment sensors, position sensors, neurological sensors, drug delivery systems, medical testing and diagnosis devices, and motion sensors.

“Electronic content” (also referred to as “content” or “digital content”) as used herein may refer to, but is not limited to, any type of content that exists in the form of digital data as stored, transmitted, received and/or converted wherein one or more of these steps may be analog although generally these steps will be digital. Forms of digital content include, but are not limited to, information that is digitally broadcast, streamed, or contained in discrete files. Viewed narrowly, types of digital content include popular media types such as MP3, JPG, AVI, TIFF, AAC, TXT, RTF, HTML, XHTML, PDF, XLS, SVG, WMA, MP4, FLV, and PPT, for example, as well as others. Within a broader approach digital content mat include any type of digital information, e.g., digitally updated weather forecast, a GPS map, an eBook, a photograph, a video, a Vine™, a blog posting, a Facebook™ posting, a Twitter™ tweet, online TV, etc. The digital content may be any digital data that is at least one of generated, selected, created, modified, and transmitted in response to a user request, said request may be a query, a search, a trigger, an alarm, and a message for example.

A “profile” as used herein, and throughout this disclosure, refers to a computer and/or microprocessor readable data file comprising data relating to settings and/or limits of an adult device. Such profiles may be established by a manufacturer/supplier/provider of a device, service, etc. or they may be established by a user through a user interface for a device, a service, or a PED/FED in communication with a device, another device, a server, or a service provider etc.

A “computer file” (commonly known as a file) as used herein, and throughout this disclosure, refers to a computer resource for recording data discretely in a computer storage device, this data being electronic content. A file may be defined by one of different types of computer files, designed for different purposes. A file may be designed to store electronic content such as a written message, a video, a computer program, or a wide variety of other kinds of data. Some types of files can store several types of information at once. A file can be opened, read, modified, copied, and closed with one or more software applications an arbitrary number of times. Typically, files are organized in a file system which can be used on numerous different types of storage device exploiting different kinds of media which keeps track of where the files are located on the storage device(s) and enables user access. The format of a file is defined by its content since a file is solely a container for data, although, on some platforms the format is usually indicated by its filename extension, specifying the rules for how the bytes must be organized and interpreted meaningfully. For example, the bytes of a plain text file are associated with either ASCII or UTF-8 characters, while the bytes of image, video, and audio files are interpreted otherwise. Some file types also allocate a few bytes for metadata, which allows a file to carry some basic information about itself.

“Metadata” as used herein, and throughout this disclosure, refers to information stored as data that provides information about other data. Many distinct types of metadata exist, including but not limited to, descriptive metadata, structural metadata, administrative metadata, reference metadata and statistical metadata. Descriptive metadata may describe a resource for purposes such as discovery and identification and may include, but not be limited to, elements such as title, abstract, author, and keywords. Structural metadata relates to containers of data and indicates how compound objects are assembled and may include, but not be limited to, how pages are ordered to form chapters, and typically describes the types, versions, relationships, and other characteristics of digital materials. Administrative metadata may provide information employed in managing a resource and may include, but not be limited to, when and how it was created, file type, technical information, and who can access it. Reference metadata may describe the contents and quality of statistical data whereas statistical metadata may also describe processes that collect, process, or produce statistical data. Statistical metadata may also be referred to as process data.

An “artificial intelligence system” (referred to hereafter as artificial intelligence, AI) as used herein, and throughout disclosure, refers to machine intelligence or machine learning in contrast to natural intelligence. An AI may refer to analytical, human inspired, or humanized artificial intelligence. An AI may refer to the use of one or more machine learning algorithms and/or processes. An AI may employ one or more of an artificial network, decision trees, support vector machines, Bayesian networks, and genetic algorithms. An AI may employ a training model or federated learning.

“Machine Learning” (ML) or more specifically machine learning processes as used herein refers to, but is not limited, to programs, algorithms, or software tools, which allow a given device or program to learn to adapt its functionality based on information processed by it or by other independent processes. These learning processes are in practice, gathered from the result of said process which produce data and or algorithms that lend themselves to prediction. This prediction process allows ML-capable devices to behave according to guidelines initially established within its own programming but evolved as a result of the ML. A machine learning algorithm or machining learning process as employed by an AI may include, but not be limited to, supervised learning, unsupervised learning, cluster analysis, reinforcement learning, feature learning, sparse dictionary learning, anomaly detection, association rule learning, inductive logic programming.

Within the following description exemplary embodiments of the invention with respect to embedded or embeddable sensors, embedded or embeddable sensor measurements, monitoring systems employing embedded or embeddable sensors, construction material properties, construction material specifications, construction material verification, construction material qualification etc. are presented and described with respect to concrete. However, it would be evident to one of skill in the art that the embodiments of the invention may be applied to other construction materials and/or other applications other than concrete and that the scope of the invention is as defined by the claims and not the exemplary embodiments of the invention described below and as depicted in the Figures.

### Embedded Sensors, Communication Networks and Connectivity

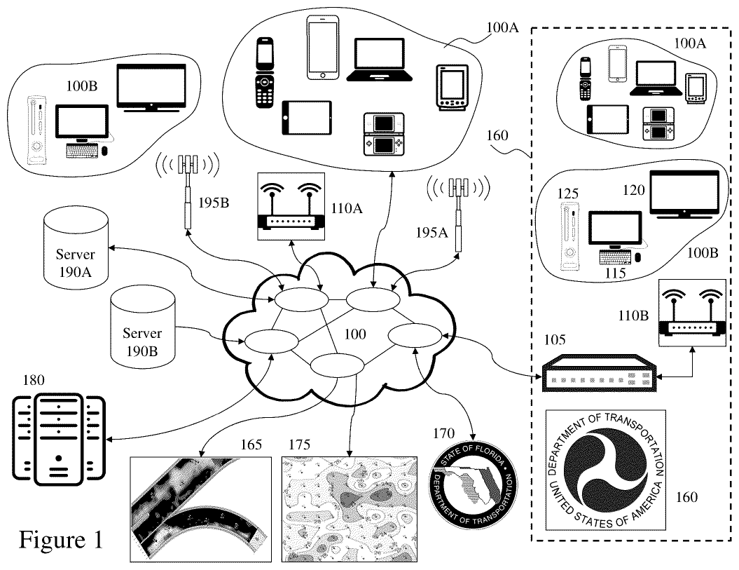

FIG. 1 depicts an exemplary network environment within which embedded sensors can be employed according to and supporting embodiments of the invention may be deployed and operate. Accordingly, as depicted first and second user groups 100A and 100B respectively interface to a telecommunications Network 100. Within the representative telecommunication architecture, a Remote Central Exchange 180 communicates with the remainder of a telecommunication service providers network via the Network 100 which may include for example long-haul OC-48/OC-192 backbone elements, an OC-48 wide area network (WAN), a Passive Optical Network, and a Wireless Link. The Remote Central Exchange 180 is connected via the Network 100 to local, regional, and international exchanges (not shown for clarity) and therein through Network 100 to first and second wireless access points (AP) 195A and 195B respectively which provide Wi-Fi cells for first and second user groups 100A and 100B, respectively. Also connected to the Network 100 are first and second Wi-Fi nodes 110A and 110B, the latter of which being coupled to Network 100 via Router 105. Second Wi-Fi node 110B is associated with Government Body 160 within which are first and second user groups 100A and 100B. Second user group 100B may also be connected to the Network 100 via wired interfaces including, but not limited to, DSL, Dial-Up, DOCSIS, Ethernet, G.hn, ISDN, MoCA, PON, and Power line communication (PLC) which may or may not be routed through a router such as router 105.

Within the cell associated with first AP 110A the first group of users 100A may employ a variety of portable electronic devices (PEDs). Within the cell associated with second AP 110B are the second group of users 100B which may employ a variety of fixed electronic devices (FEDs).

Also connected to the Network 100 are first and second APs 195A and 195B respectively which provide, for example, cellular GSM (Global System for Mobile Communications) telephony services as well as 3G and 4G evolved services with enhanced data transport support. Second AP 195B provides coverage in the exemplary embodiment to first and second user groups 100A and 100B. Alternatively the first and second user groups 100A and 100B may be geographically disparate and access the Network 100 through multiple APs, not shown for clarity, distributed geographically by the network operator or operators. First AP 195A as show provides coverage to first user group 100A and Government Body 160, which comprises second user group 100B as well as first user group 100A. Accordingly, the first and second user groups 100A and 100B may according to their particular communications interfaces communicate to the Network 100 through one or more wireless communications standards such as, for example, IEEE 802.11, IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, and IMT-2000. It would be evident to one skilled in the art that many portable and fixed electronic devices may support multiple wireless protocols simultaneously, such that for example a user may employ GSM services such as telephony and SMS and Wi-Fi/WiMAX data transmission, VOIP and Internet access. Accordingly, portable electronic devices within first user group 100A may form associations either through standards such as IEEE 802.15 or Bluetooth as well in an ad-hoc manner.

Also connected to the Network 100 are Concrete Analysis Environment 165, State Body 170, and Building Analysis Environment 175 as well as first and second servers 190A and 190B which together with others not shown for clarity, may host according to embodiments of the inventions multiple services associated with one or more organizations, including but not limited to, a provider of the software operating system(s) and/or software application(s) associated with embeddable sensors, a provider of embeddable sensors, provider of one or more aspects of wired and/or wireless communications, provider of the electrical measurement devices, provider of mapping analysis software, provider of electrical measurement analysis software, global position system software, materials databases, building databases, regulatory databases, license databases, construction organizations, websites, and software applications for download to or access by FEDs, PEDs, embeddable sensors and electrical measurement systems. First and second Servers 190A and 190B may also host for example other Internet services such as a search engine, financial services, third party applications and other Internet based services.

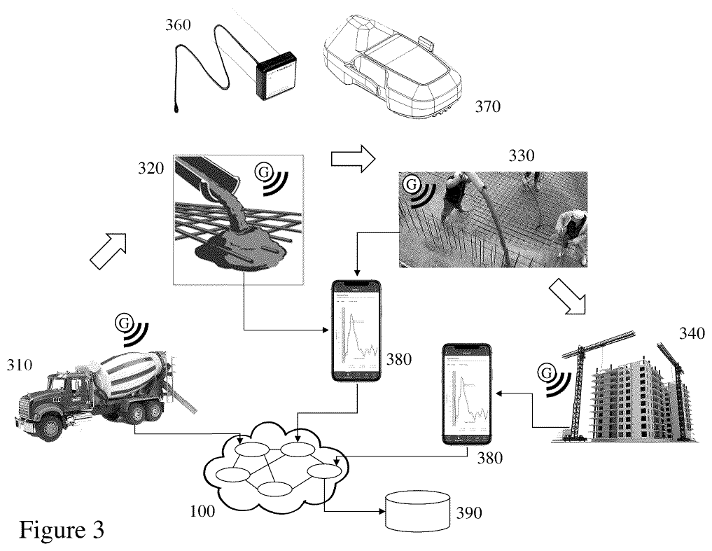

Accordingly, it would be evident to one skilled in the art that embeddable sensors according to embodiments of the invention described in respect of FIGS. 3 to XX may be connected to a communications network such as Network 100 either continuously or intermittently. It would be further evident that the measurement data from the embeddable sensors may be employed to provide information to a construction material manufacturer, construction companies, engineering companies, infrastructure owners, regulators, etc. This measurement data with or without additional processing may be employed for quality assurance, compliance to construction specifications, for the construction material manufacturer, construction companies, engineering companies, infrastructure owners, etc. as well as a result of activities triggered by, for example, the Government Body 160 and/or State Body 170 in order to address regulatory requirements, safety concerns etc. Accordingly, the engineers, workers and/or technicians who will be performing the measurements may be able to access Concrete Analysis Environment 165 to obtain construction material manufacturing data, deployment data, performance data etc. and/or Building Analysis Environment 175 to obtain architect drawings, engineering data, design data, etc. relating to the concrete structure being assessed. Concrete Analysis Environment 165 and/or Building Analysis Environment 175 may be provided, for example, by a manufacturer of embeddable sensors, a service provider, a construction material manufacturer, construction material company, or a third-party service provider.

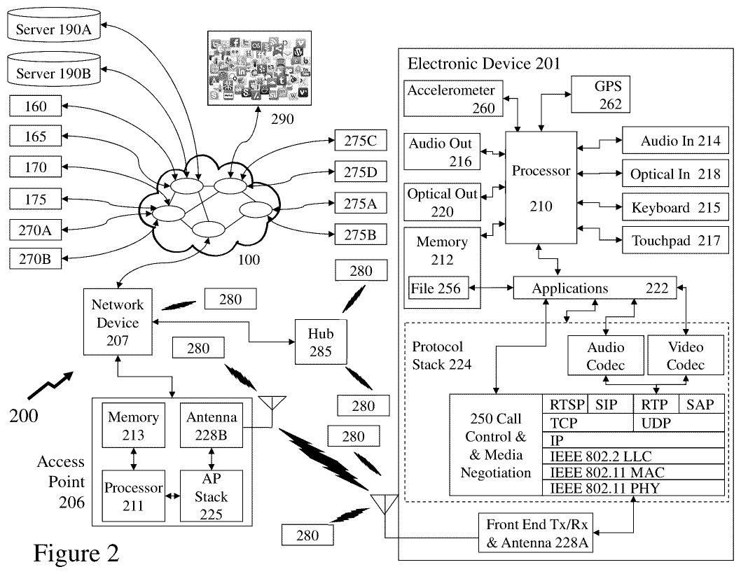

FIG. 2 depicts an exemplary electronic device supporting communications to a network such as depicted in FIG. 1 and with embedded sensors according to and supporting embodiments of the invention. Within schematic 200 the Electronic Device 201 supporting Embedded (or Embeddable) Sensor (MBEDSEN) Systems, Applications and Platforms (SAPs) and EMBSEN-SAP features according to embodiments of the invention is connected. Electronic Device 201 may, for example, be a PED, a FED, or a wearable device and may include additional elements beyond those described and depicted. Also depicted in conjunction with the Electronic Device 201 are exemplary internal and/or external elements forming part of a simplified functional diagram of an Electronic Device 201 within an overall simplified schematic of a system supporting EMBSEN-SAP features according to embodiments of the invention which include includes an Access Point (AP) 206, such as a Wi-Fi AP for example, a Network Device 207, such as a communication server, streaming media server, and a router. The Network Device 207 may be coupled to the AP 206 via any combination of networks, wired, wireless and/or optical communication links. Also connected to the Network 100 are Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B, respectively.

As depicted the Electronic Device 201 may communicate directly with AP 206 as well as with embeddable and/or embedded sensors (MBEDSENs) 280, such as Giatec Scientific's SmartRock™ and BlueRock™ sensor devices for example, wherein MBEDSENs 280 may be embedded into construction materials at various points in their life cycle such as during their manufacture, deployment, and post-deployment. The inventors describing several MBEDSEN 280 concepts within patent applications such as
U.S. Provisional Patent Application 63/199,901 and its subsequent World Patent Application PCT/CA2021/050141;
U.S. Provisional Patent Application 62/828,585 and its subsequent World Patent Application PCT/2020/050440 together with Canadian, European and United States national phase applications, divisions and continuations up to and including CA 3,132,214; EP 20783564.6 and U.S. Ser. No. 17/593,964;
U.S. Provisional Patent Application 62/866,188 and its subsequent World Patent Application PCT/CA2019/000057 together with Canadian, European and United States national phase applications, divisions and continuations up to and including CA 3,099,005; EP 3,788,353; and US 2021/0,063,336;
U.S. Provisional Patent Application 62/315,202 and its subsequent Canadian, European and United States national phase applications, divisions and continuations up to and including CA 3,099,362; EP 3,236,258 and US 2021/0,382,032;
U.S. Provisional Patent Application 61/992,364 and its subsequent World Patent Application PCT/CA2015/000,314 together with Canadian, European and United States national phase applications, divisions and continuations up to and including CA 3,125,171; EP 3,869,187 and US 2020/0,408,707; and
U.S. Provisional Patent Application 61/758,309 and its subsequent direct formal and continuation patents up to and including U.S. Patent Application 2021/0,033,553;

An MBEDSEN 280 may communicate as depicted (although other configurations not described are supported by embodiments of the invention) by one or more of:
directly with Electronic Device 201;
to Electronic Device 201 via a Wireless Booster/Repeater such as described below in respect of embodiments of the invention;
to a Hub 285 which acquires data from MBEDSENs 280 and pushes it to an endpoint, such a first Server 190A for example, via a Network Device 207 (which may be integrated with the Hub 285) through wired or wireless link(s);
to an AP 206; and
to a Network Device 207.

An Electronic Device 201 includes one or more Processors 210 and a Memory 212 coupled to Processor(s) 110. AP 206 also includes one or more Processors 211 and a Memory 213 coupled to Processor(s) 210. A non-exhaustive list of examples for any of Processors 210 and 211 includes a central processing unit (CPU), a digital signal processor (DSP), a reduced instruction set computer (RISC), a complex instruction set computer (CISC), a graphics processing unit (GPU) and the like. Furthermore, any of Processors 210 and 211 may be part of application specific integrated circuits (ASICs) or may be a part of application specific standard products (ASSPs). A non-exhaustive list of examples for Memories 212 and 213 includes any combination of the following semiconductor devices such as registers, latches, ROM, EEPROM, flash memory devices, non-volatile random access memory devices (NVRAM), SDRAM, DRAM, double data rate (DDR) memory devices, SRAM, universal serial bus (USB) removable memory, and the like.

Electronic Device 201 may include an audio input element 214, for example a microphone, and an Audio Output Element 216, for example, a speaker, coupled to any of Processor(s) 210. Electronic Device 201 may include an Optical Input Element 218, for example, a video camera or camera, and an Optical Output Element 220, for example an LCD display, coupled to any of Processor(s) 210. Electronic Device 201 may also include a Keyboard 215 and Touchpad 217 which may for example be a physical keyboard and touchpad allowing the user to enter content or select functions within one of more Applications 222. Alternatively, the Keyboard 215 and Touchpad 217 may be predetermined regions of a touch sensitive element forming part of the display within the Electronic Device 201. The one or more Applications 222 that are typically stored in Memory 212 are executable by any combination of Processor(s) 210. Electronic Device 201 also includes Accelerometer 260 providing three-dimensional motion input to the Processor(s) 210 and GPS 262 which provides geographical location information to Processor(s) 210.

Electronic Device 201 includes a Protocol Stack 224 and AP 206 includes an AP Stack 225. Within Protocol Stack 224 is shown an IEEE 802.11 protocol stack but alternatively may exploit other protocol stacks such as an Internet Engineering Task Force (IETF) multimedia protocol stack for example or another protocol stack. Likewise, AP Stack 225 exploits a protocol stack but is not expanded for clarity. Elements of Protocol Stack 224 and AP Stack 225 may be implemented in any combination of software, firmware and/or hardware. Protocol Stack 224 includes an IEEE 802.11-compatible PHY module that is coupled to one or more Tx/Rx & Antenna Circuits 228A and an IEEE 802.11-compatible MAC module which is coupled to an IEEE 802.2-compatible LLC module. Protocol Stack 224 also includes modules for Network Layer IP, a transport layer User Datagram Protocol (UDP), a transport layer Transmission Control Protocol (TCP), a session layer Real Time Transport Protocol (RTP), a Session Announcement Protocol (SAP), a Session Initiation Protocol (SIP) and a Real Time Streaming Protocol (RTSP). Protocol Stack 224 includes a presentation layer Call Control and Media Negotiation module 250, one or more audio codecs and one or more video codecs. Applications 222 may be able to create maintain and/or terminate communication sessions with the Network Device 207 by way of AP 206 and therein via the Network 100 to one or more of Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B respectively.

Typically, Applications 222 may activate any of the SAP, SIP, RTSP, and Call Control & Media Negotiation 250 modules for that purpose. Typically, information may propagate from the SAP, SIP, RTSP, Call Control & Media Negotiation 250 to the PHY module via the TCP module, IP module, LLC module and MAC module. It would be apparent to one skilled in the art that elements of the Electronic Device 201 may also be implemented within the AP 206 including but not limited to one or more elements of the Protocol Stack 224, including for example an IEEE 802.11-compatible PHY module, an IEEE 802.11-compatible MAC module, and an IEEE 802.2-compatible LLC module. The AP 206 may additionally include a network layer IP module, a transport layer User Datagram Protocol (UDP) module and a transport layer Transmission Control Protocol (TCP) module as well as a session layer Real Time Transport Protocol (RTP) module, a Session Announcement Protocol (SAP) module, a Session Initiation Protocol (SIP) module and a Real Time Streaming Protocol (RTSP) module, and a call control & media negotiation module. Portable electronic devices (PEDs) and fixed electronic devices (FEDs) represented by Electronic Device 201 may include one or more additional wireless or wired interfaces in addition to or in replacement of the depicted IEEE 802.11 interface which may be selected from the group comprising IEEE 802.15, IEEE 802.16, IEEE 802.20, UMTS, GSM 850, GSM 900, GSM 1800, GSM 1900, GPRS, ITU-R 5.138, ITU-R 5.150, ITU-R 5.280, IMT-1010, DSL, Dial-Up, DOCSIS, Ethernet, G.hn, ISDN, MoCA, PON, and Power line communication (PLC).

The Front End Tx/Rx & Antenna 228A wirelessly connects the Electronic Device 201 with the Antenna 128B on Access Point 206, wherein the Electronic Device 201 may support, for example, a national wireless standard such as GSM together with one or more local and/or personal area wireless protocols such as IEEE 802.11 a/b/g Wi-Fi, IEEE 802.16 WiMAX, and IEEE 802.15 Bluetooth for example. Accordingly, it would be evident to one skilled the art that the Electronic Device 201 may accordingly download original software and/or revisions for a variety of functions. In some embodiments of the invention the functions may not be implemented within the original as sold Electronic Device 201 and are only activated through a software/firmware revision and/or upgrade either discretely or in combination with a subscription or subscription upgrade for example. Accordingly, as will become evident in respect of the description below the Electronic Device 201 may provide a user with access to one or more EMBSEN-SAPs including, but not limited to, software installed upon the Electronic Device 201 or software installed upon one or more remote systems such as those associated with Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B respectively.

Accordingly, within the following description a remote system/server may form part or all of the Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B respectively. Within the following description a local client device may be Electronic Device 201 such as a PED, FED or Wearable Device and may be associated with one or more of the Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B respectively. Similarly, a storage system/server within the following descriptions may form part of or be associated within Social Media Networks (SOCNETS) 290; Government Body 160, Concrete Analysis Environment 165, State Body 170, Building Analysis Environment 175; first and second remote systems 270A and 270B respectively; first and second websites 275A and 275B respectively; first and second 3rd party service providers 275C and 275D respectively; and first and second Servers 190A and 190B respectively.

Embedded Sensors within Construction Material Life Cycle

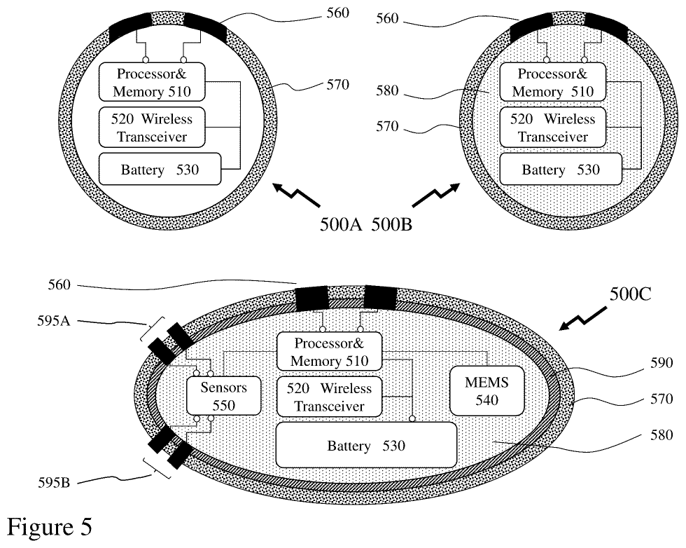

FIG. 3 depicts an embedded sensor methodology for data logging concrete properties from initial mix through pouring, curing, and subsequently according to embodiments of the invention. As such MBEDSENs, such as first to third MBEDSENs 500A to 500C in FIG. 5, depicted in first configuration 360 and second configuration 370 in FIG. 2, may be added to a construction material batch, e.g., concrete which is loaded onto a concrete truck at the batching plant, within an embodiment of the invention. It is therefore possible to “tag,” i.e., load into, the MBEDSEN information relevant to the mix as well as delivery data etc. This information as well as other measurements made by the MBEDSENs during the transportation, delivery, deployment, etc. can be accessed by a wireless interface by an end user. According to the design of the MBEDSEN the lifetime of the MBEDSEN may be short, e.g., a few days or weeks, moderate, e.g., a few months, or long, e.g., years.

As such the tagging of the MBEDSENs may include, but not be limited to, information such as batch identity, truck identity, date, time, location, batch mix parameters, etc. but also importantly information such as the maturity calibration curves for the mix established by the material manufacturer. Accordingly, depending upon the degree of complexity embedded into the MBEDSEN such data may be either retrieved for remote storage and subsequent use or it may be part of the MBEDSENs processing of electrical measurement data such that calibration data of the construction material is already factored into the data provided by the MBEDSENs. Accordingly, the MBEDSENs, such as first configuration 360 and second configuration 370 may be added to the construction material, e.g., concrete, at the batching point 310 either tagged already or tagged during loading. Subsequently upon delivery and pouring 320 the MBEDSENs may be read for information regarding the delivery process etc.

Once poured the MBEDSENs may be read for curing information 330 and then subsequently, depending upon the battery-power consumption etc., periodically read for lifetime data 340 of the concrete. In each instance the acquired data may be acquired wirelessly and stored on a user's PED or it may then be pushed to a Network 100 and therein to one or more servers 390. For devices wireless interrogating the MBEDSENs these may be executing a software application which presents to the user concrete parameter data either as provided from the MBEDSEN(s) directly using the calibration curves stored within or upon the device using calibration curve data stored within the MBEDSEN but not processed by it, stored within the device, or retrieved from the data stored upon the remote server 390.

As depicted first configuration 260 is enabled when an electrical circuit is completed via the flying leads. In second configuration 270 the sensor may be enabled through a wireless signal, a vibration exceeding a threshold, via an electrical circuit being completed upon removal of a sensor cable which incorporates a magnetic element within the sensor head removed from a housing within the second configuration 270 MBEDSEN, increase in humidity beyond a threshold, decrease in light, etc. Accordingly, the embodiments of the invention support tagging the sensors and embedding the maturity calibration curves in the sensor. These curves may for example be mix-specific and depending on the temperature history of the concrete can be used to estimate the strength of the construction material, e.g., concrete. By embedding them within the sensors and the sensors employing this data the construction material manufacturer does not need to release commercially sensitive information such as their proprietary mix and calibration curves.

Based upon the combination of MBEDSENs within the construction material and their wireless interrogation and mobile/cloud-based software applications other technical enhancements may be implemented, including for example:
Weather forecast API, such that the ambient temperature prediction in conjunction with current construction material data can be used to predict/project the strength identifying quality problems earlier;
Automatic detection of construction material pouring time, e.g., from electrical connection once the concrete is poured or change in the pressure, humidity, light etc.;
Tagging the sensor using NFC with smartphone;
Data integrity and management on remote servers;
Data analytics and/or artificial intelligence on data analysis as the MBEDSEN manufacturer may acquire data from a large number of job sites allowing additional analytics, reporting, alarms etc.;
AN MBEDSEN manufacturer may establish so-called “big data” on construction material properties and construction material curing cycles/processes across a large number of job sites, geographic regions, time frames etc. allowing them to provide feedback from their server-based processes to the end user;
Push notifications, such as for example the formwork company is notified when it is time to remove the formwork based upon actual concrete curing data; and
Heat optimization wherein for example closed loop feedback of the temperature history and strength development can be employed to optimize heating employed in cold climates to ensure the concrete slabs gain sufficient strength within a specific period.

In addition to measuring, for example, temperature, DC electrical conductivity, and AC electrical conductivity it would be evident that additional parameters as discussed and described supra in respect of embodiments of the invention may be measured and monitored, including, but not limited to, concrete moisture content, concrete internal relative humidity, concrete pH, concrete mixture consistency, concrete workability (slump), concrete air content, hydraulic pressure, segregation, cracking, penetration of external ions into concrete, dispersion of fibers, and dispersion of chemical additives and efficacy/reactivity of supplementary cementitious materials.

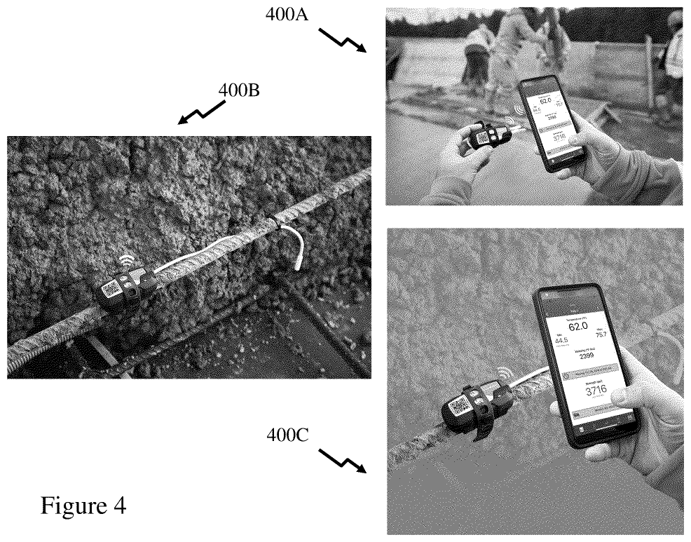

FIG. 4 depicts a sensor according to the embodiment of the invention prior to deployment, in deployed state and being read in deployed state in first to third views 400A to 400C, respectively. Referring to first view 400A a user is depicted wirelessly communicating from a smartphone, although other PEDs may be employed. Initially, the user may acquire an image of the barcode on the sensor establishing the unique identity of the sensor such that their PED can establish communications to that sensor discretely even in the presence of multiple sensors within wireless range of the PED (or FED within other embodiments of the invention). Once wireless communications are established with the sensor the user's PED may transfer information to the sensor and/or acquire information from the sensor. In these instances, the sensor detects wireless communications and awakes from a sleep state. Within other embodiments of the invention a sensor element, e.g., temperature sensor, may be stored within an opening within the sensor wherein removal of the sensor element from the opening within the sensor triggers the sensor to awake as removal from its initial shipped state with the sensor element deployed within the opening within the sensor body would indicate imminent deployment.

Accordingly, once data has been acquired from and/or provided to the sensor it is deployed and concrete pouring begins. As depicted in second view 400B the sensor is attached to a rebar with a strap and a sensor cable which terminates at the distal end from the sensor with a sensor head comprising a sensor element, is also tied to the rebar for part of its length according to how the sensor head will be deployed, e.g., how far below the rebar. As depicted in third view 400C the sensor can be subsequently wirelessly communicated with to retrieve sensor data, material characteristics, etc. even when buried in the wet concrete. Second and third views 400B and 400C showing only partial concrete pouring to allow the rebar and sensor to be visible.

### Exemplary Embedded Sensor Geometries

Now referring to FIG. 5 there are depicted first to third MBEDSENs 500A to 500C according to embodiments of the invention. Referring to first MBEDSEN 500A contacts 560 are formed within outer Shell 570 defining an interior within which are disposed a processor with associated memory 510 (hereinafter, processor). The processor 510 being coupled to a Wireless Transceiver 520 and a Battery 530. Accordingly, electrical conductivity (for example) between the contacts 560 may be monitored (e.g., arising from water within a concrete mix), processed with the processor 510, stored and then subsequently transmitted via Wireless Transceiver 520 when a link is established to a portable electronic device (PED) such as smartphone, tablet PC, or dedicated device. The Shell 570 may be formed from a variety of materials, including but not limited to, metals (from which the contacts are isolated by insulating rings etc.), ceramics (e.g. alumina, zirconia, etc.), composites (e.g. fiber reinforced polymer, ceramic matrix composites, concrete, glass-reinforced plastic) and plastics (e.g. short-fiber thermoplastics, long-fiber thermoplastics, thermosetting plastics, filled plastics, synthetic rubber, elastomer, etc.).

Second MBEDSEN 500B depicts essentially the same construction as MBEDSEN 500A except that the interior of the shell is now filled with a Filler 580. Second filler material 580 may be a resilient Filler 580 surrounded by a soft Shell 570 such as synthetic rubber or elastomer, for example, or alternatively the Filler 580 may be semi-resilient in combination with a resilient Shell 570. Such semi-resilient fillers 580 may include thermosetting resins, catalyzed resins, cured silicone gels, etc. used in conjunction with a Shell 570 formed from a plastic or rubber, for example.

Third MBEDSEN 500C exploits the same Filler 580 with Shell 570 but now an intermediate Casing 590 is disposed between the outer Shell 570 and the inner Filler 580. For example, Casing 590 may be an impermeable membrane, e.g., Gore-Tex™, that limits moisture ingress to the MBEDSEN 500C but allows air or gas permeability. Further, MBEDSEN 500C now comprises in addition to the processor 510, Wireless Transceiver 520, and Battery 530 additional sensors 560 which are coupled to first and second SENsor INTerfaces (SENINTs) 590A and 590B which together with contacts 560 provide external sensing data to the processor 510. Further a microelectromechanical system (MEMS) 540 within the MBEDSEN 500C provides data to the processor 510 wherein the MEMS 540 may comprise, for example, an accelerometer such as a one-dimensional (1D), two-dimensional (2D) or three-dimensional (3D) accelerometer providing data relating to motion, shock, etc. Within different embodiments of the invention some SENSINTs may have direct exposure to the external environment whereas others may be indirect or via a barrier material etc. or have a characteristic that varies in response to an external environmental aspect. Sensors may include, but are not limited to, temperature, electrical resistance, pressure, light, acceleration (e.g. MEMS accelerometer), vibration (e.g. MEMS sensor), humidity (e.g. capacitive sensor barriered with a vapour barrier to prevent direct fluid contact), pH (e.g. ion sensitive field effect transistor-ISFET pH sensor), ion content (to detect externally penetrating chemicals or materials), chloride content, microphone or acoustic sensor (to detect crack propagation), gas sensor (e.g. nitrogen, oxygen to detect air within cracks propagating to the surface of the concrete), corrosion detectors, visible optical sensors, ultraviolet optical sensors, and infrared optical sensors. More advanced sensors may provide dedicated hardware, functionality, and software to enable more advanced techniques such as nuclear magnetic resonance, electrochemical, X-ray diffraction, optical spectrometry, thermogravimetric analysis, a half cell, etc. as well as corrosion resistance etc.

### Construction Material Specification Definition, Verification, Optimization and Service Life Assessment Including Machine Learning/Artificial Intelligence Based Methods

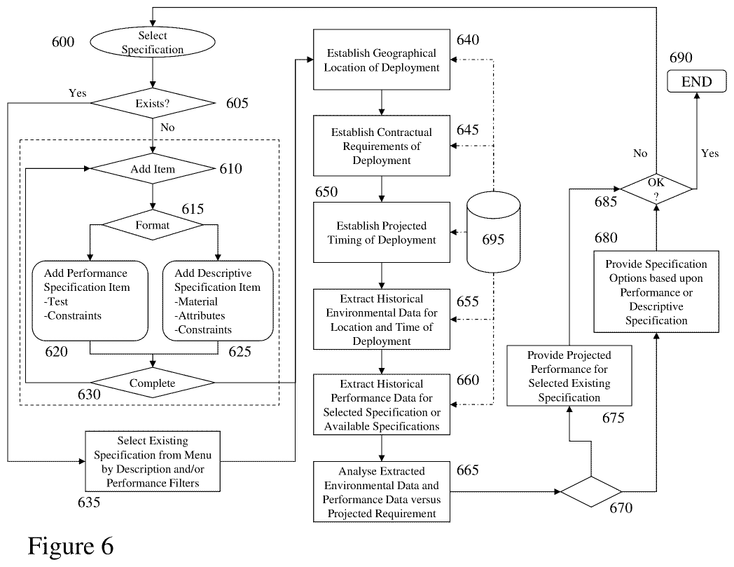

In order to purchase concrete, the typical prior art method is through a phone inquiry to a ready-mix concrete producer. The producer receives information from the user on the desired properties (e.g. 3-day strength or a slump/flowability criteria) and chooses a mixture that best-matches these properties. The purchaser typically only performs a few inquiries and may not reach the best option in terms of cost or performance. This can be solved using an online “marketplace” for concrete wherein the purchaser enters extended data to their application, deployment scenario etc. Accordingly, a purchaser may exploit an exemplary process flow such as depicted in FIG. 6 for the establishment of a manufacturing specification in respect of a construction material, such as concrete for example, exploiting acquired performance data from SMAKs and/or other performance monitoring sensors. Accordingly, the process as depicted comprises steps 600 to 690 in conjunction with a database 695.

Step 600 wherein the user initiates the process for selecting a material specification.

Step 605 wherein a determination is made as to whether the specification already exists or not, wherein if the specification exists the process proceeds to step 635 otherwise it proceeds to step 610.

Steps 610 to 630 wherein the process of construction of a particular material specification containing a number of items is presented. Upon addition of an item through steps 610 to 625 the process determines in step 610 whether the specification is complete or not and proceeds to step 640 upon completion or step 610 if not. Within some embodiments of the invention the determination of whether the completion has occurred is based upon selecting a number of items until a total number items desired is achieved. Optionally, the determination is made by the user or through a combination of the process and user. For example, the user may be guided to choose a base material (e.g. type of cement), a number of additives in predetermined classes of additive (e.g. aggregate, admixture, etc.) wherein selection of at least one in each as the process moves sequentially from one to another class would mean completion of the specification. Accordingly, the process will loop until the appropriate number of specification items are defined and/or the user denotes completion.

An initial decision is made in step 615 as to whether the specification item to be created will be prescriptively based or performance based. A prescriptive specification item may reference a specific material or materials and the materials attributes and/or constraints while a performance-based specification item would be established through the physical and/or chemical characteristics of the construction material either after completion of production or upon installation and thereafter. Accordingly, these are performed in steps 620 and 625. In this manner the construction material may be specified in terms of final target performance rather than by specific brand, identity and/or composition. Within this series of steps 610 to 630 the user may also establish one or more quantifiable properties and/or standard tests and may include predetermined dependent variables and/or constraints of which the construction material must satisfy. These would typically be provided to the user from a database such as database 695. Where the specification items are listed descriptively then the item may include the material and its material quantifiable property or properties such as water/cement ratio, a set of material attributes, and/or constraints which the materials should fall within.

Once defined, either descriptively or by performance, the specification item is preferably complete and added to the concrete specification being built. The list of completed specification items may be compared to the total number of items that are to be defined for the current specification and if all of the items have not been completed, the next specification item should be defined. Each additional item can be either descriptive or performance-based again and a concrete specification may therefore contain a mix of both descriptive and performance-based specification items. Once all of the items for a particular concrete specification have been properly defined and constrained the specification is stored.

Step 635 wherein if the decision in step 605 was to select an existing specification then the user proceeds to make the selection from a menu using description and/or performance filters, for example.

Step 640 wherein upon selection of the established specification or completion of the new specification the process establishes the geographical location for the deployment of the construction material. This may, for example, be by user entry or alternatively through means such as association of the construction material specification to a project wherein the data for the project includes this and other information as required including, but not limited to, that in steps 645 to 660. Alternatively, the user's location may be established in dependence upon an electronic address, e.g. an Internet Protocol Address, and hostname in a manner similar to that employed in geo-targeting advertisements to users upon PEDs and/or FEDs.

Step 645 wherein the contractual requirements associated with the deployment are established. These may, for example, be a restriction on how long formwork can be left up after construction material is poured, how much material is required, time limits for delivery and pouring as the location may be within a busy downtown core, an issue from another aspect of the project etc.

Step 650 wherein projected timing of the project is established such as when formwork will be established, when pouring should be started, when pouring should be complete, etc. are extracted from the database 695

Step 655 wherein historical data relating to the location and the projected time of deployment are extracted from the database 695.

Step 660 wherein historical performance data for the selected specification or available specifications based upon the performance and/or descriptive specification items is extracted from the database 695.

Step 665 wherein the extracted historical data relating to location, time, historical environmental data, historical performance data etc. are processed to establish a projected set of construction material characteristics at one or more predetermined points in time.

Step 670 wherein the process determines whether the user selected an existing specification and proceeds to step 675 or provided specification options and proceeds to step 680.

Step 675 wherein the user is provided with projected performance of the selected existing specification based upon the location, time, historical environmental data, historical performance data etc.

Step 680 wherein the user is provided with specification options based upon the target characteristics defined by the performance and/or specification items selected by the user being matched against the available construction material specifications based upon the location, time, historical environmental data, historical performance data etc.

Step 685 wherein the user determines whether to stop the process wherein the process proceeds to step 690 or to iterate and the process returns to step 600. Optionally, in the subsequent iterations the user may be provided with options to adjust the project related data such as whether a deployment is undertaken earlier or later, whether an additive should be employed, etc.

Optionally, the process automatically performs the determination in step 685 based upon the projected performance meeting the required performance requirements. Optionally, the process may extract the target performance specification items from the database 695 based upon selection of the project by the user within another process step and therein perform a construction material selection automatically.

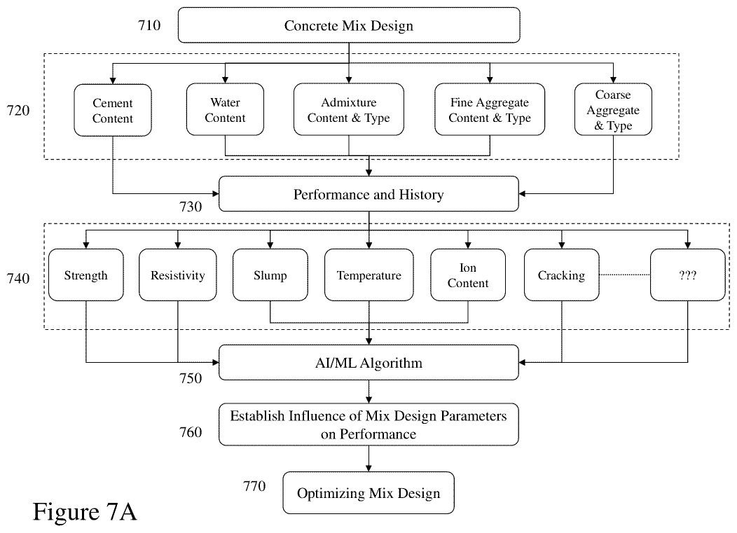

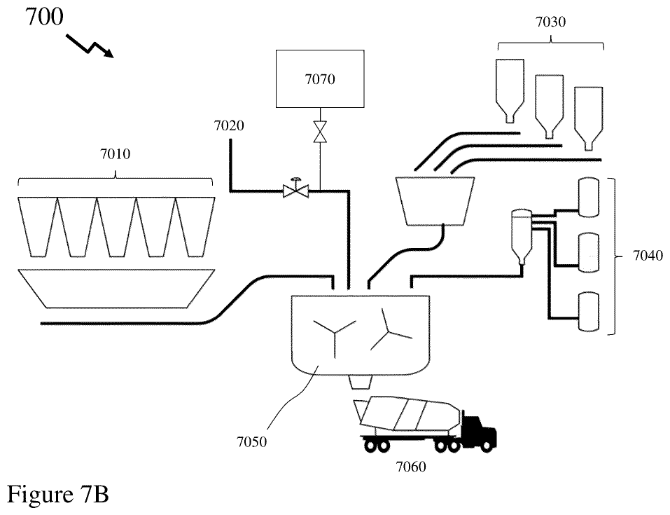

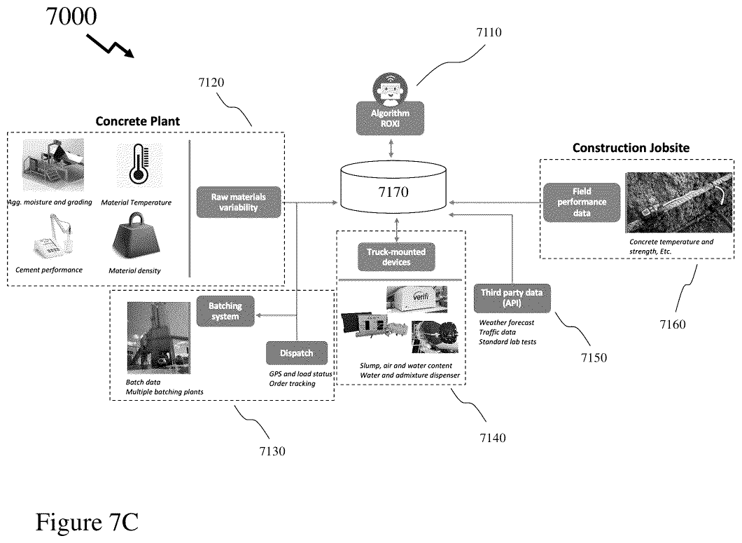

It would also be evident that the acquisition of data relating to multiple construction materials, e.g. a concrete mix, also allows for optimization of a concrete mix as a discrete process for a manufacturer as opposed to the determination of a mix design for a specific project as described and depicted in FIG. 7A. Such an exemplary process flow is depicted in FIG. 7A for optimizing a manufacturing specification for a construction material according to an embodiment of the invention exploiting machine learning and artificial intelligence comprising first to seventh blocks 710 to 770 respectively, these being:
First block 710 wherein a user can select a concrete mix design;
Second block 720 wherein the concrete mix elements are established such as cement content, water content, admixture content and type, fine aggregate content and type, and coarse aggregate content and type;
Third block 730; wherein the performance data and history for the selected mix are extracted from the stored data within the remote servers which can comprise the data acquired from embedded sensors, partially embedded sensors, third-party sources such as environmental data etc., as well as data established at the time of concrete mix production and transportation;
Fourth block 740 wherein the extracted performance data and history are analysed to extract different properties of the concrete such as strength, resistivity, slump, temperature, ion content, cracking etc.
Fifth block 750 wherein artificial intelligence (AI)/machine learning (ML) algorithms and/or processes are employed to process the extracted data;
Sixth block 760 wherein the analysis performed by the AI/ML algorithms is assessed to establish the influence of mix design parameters on the performance of the concrete mix as variations in mix preparation, mix transportation, deployment, life cycle etc. can be determined and/or evaluated; and
Seventh block 770 wherein amendments to the concrete mix can be determined to optimize the mix such as for improved long term strength, reduced chloride ions, reduced time before formwork removal, reduced impact of ambient environment etc.

The process described and depicted in respect of FIG. 7A may be fully automated or it may require user input such as identification of which aspects of performance of the mix are to be assessed/optimized. Further, the analysis may be filtered such as for geographic location, season, type of infrastructure element, etc.

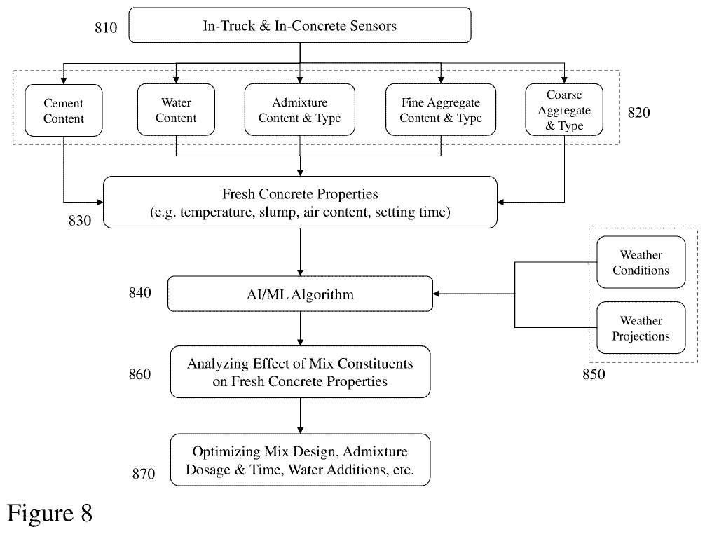

It would be evident that the exemplary process depicted in FIG. 7A with respect to second Block 720, relating to concrete mix elements (such as cement content, water content, admixture content and type, fine aggregate content and type, and coarse aggregate content and type for example), and fourth Block 740, relating to extracted performance data and history (such as strength, resistivity, slump, temperature, ion content, and cracking for example) may exploit data acquired during different portions of the construction material life cycle such as production, delivery, deployment, and ongoing life post-completion of the construction phase. For example, according to embodiments of the invention as described below FIGS. 8 to 10 depict exemplary process flows for optimizing a construction material during transportation, for reduced cost/waste, and for service life assessment.

However, it would be evident that other process flows for optimizing one or more aspects of the construction material life cycle and/or raw material(s) employed within the construction material may exploit data acquired from sensors embedded within the construction material as well as other sensors associated with different life cycle points may be employed according to or exploiting embodiments of the invention exploiting machine learning and artificial intelligence. For example, referring to FIG. 7B there is depicted an exemplary schematic of extended sensor integration within a concrete production facility in conjunction with embedded sensors monitoring one or more portions of a subsequent life cycle of the construction material for optimizing a mixture for a construction material according to an embodiment of the invention.

Within FIG. 7B there is depicted a schematic of a concrete plant (Plant 700) (also known as a concrete mixing plant or concrete batching plant) wherein additional sensors are incorporated to provide additional data to embodiments of the invention exploiting machine learning and artificial intelligence, including but not limited to, those depicted in FIGS. 6, 7A and 8-10 respectively for concrete or other construction materials.

Accordingly, within Plant 700 of FIG. 7B a Truck 7060 is depicted to transport a concrete mixture (hereinafter a “mix”) produced by Mixer 7050 according to a mixture specification (mix specification). The Mixer 7050 being fed with the raw materials comprising, for example, from one or more Aggregates 7010, Waster 7020, and Cement 7030. Optionally, one or more binders or admixtures may be added from further storage Binding and Admixtures 7040 may be added at this initial mixing rather than subsequently, such as described and depicted in respect of FIG. 8 for example. Multiple silos may be provided within Aggregates 7010 to store different aggregates, aggregate grades, etc. whilst Cement 7030 may similarly store one or more cements within multiple silos. In addition to the Water 7020 a Recycled Water 7070 storage tank may be provided which is fed from the Mixer 7050 or a stage after the Mixer 7050 wherein the linkage(s) to the Recycled Water 7070 from the Mixer 7050 and/or other stages are not depicted for clarity.

Accordingly, within an embodiment of the invention the Plant 700 may comprise one or more sensors in addition to the exploitation of embedded sensors within one or more of the Aggregates 7010, Water 7020, Cement 7030, and Admixtures 7040 or added to the Mixer 7050 at the production of the batch of construction material to be provided to the Truck 7060. These additional sensors may include, but not be limited to:
Aggregate Sensors:
   Temperature sensor(s) per silo (and/or feed to Mixer 7050) to monitor temperature of the aggregate during storage and/or delivery to the Mixer 7050;
   Moisture sensor(s) per silo (and/or feed to Mixer 7050) to monitor moisture level(s) of the aggregate(s) during storage and/or delivery to the Mixer 7050;
Water Sensor(s):
   Temperature sensor(s) to monitor temperature of the fresh water, Water 7020, during delivery to the Mixer 7050;
   Chemical sensor(s) to monitor one or more chemicals to determine their presence and/or level during delivery of the fresh water, Water 7020, to the Mixer 7050;
   Ion sensor(s) to monitor one or more ionic species to determine their presence and/or level during delivery of the fresh water, Water 7020, to the Mixer 7050;
Binder and Admixture Sensors:
   Temperature sensor(s) to monitor temperature of the binder(s) and/or admixture(s), Binders and Admixtures 7040, during storage and/or delivery to the Mixer 7050;
   Chemical sensor(s) to monitor one or more chemicals to determine their presence and/or level within the binder(s) and/or admixture(s), Binders and Admixtures 7040, during storage and/or delivery to the Mixer 7050;
   Ion sensor(s) to monitor one or more ionic species to determine their presence and/or level within the binder(s) and/or admixture(s), Binders and Admixtures 7040, during storage and/or delivery to the Mixer 7050;
Mixer Sensor(s):
   Temperature sensor(s) to monitor temperature of the mixture during mixing and/or delivery to the Truck 7060;
   Chemical sensor(s) to monitor one or more chemicals to determine their presence and/or level during mixing and/or delivery of the mixed mixture to the Truck 7060;
   Ion sensor(s) to monitor one or more ionic species to determine their presence and/or level during mixing and/or delivery of the mixed mixture to the Truck 7060;
   Moisture sensor(s) to monitor the moisture level during mixing and/or delivery of the mixed mixture to the Truck 7060;
Recycled Water Sensor(s):
   Temperature sensor(s) to monitor temperature of the recycled water, Recycled Water 7070, during delivery to the Mixer 7050;
   Chemical sensor(s) to monitor one or more chemicals to determine their presence and/or level during delivery of the recycled water, Recycled Water 7070, to the Mixer 7050;
   Ion sensor(s) to monitor one or more ionic species to determine their presence and/or level during delivery of the recycled water, Recycled Water 7070, to the Mixer 7050; and
Density sensor(s) to monitor the density of the recycled water, Recycled Water 7070, to the Mixer 7050 wherein the density can be employed to establish a level of additional solids present within the recycled water fed to the Mixer 7050.

Now referring to FIG. 7C there is depicted a schematic System 7000 of an overall system according to embodiments of the invention for providing continuous mix design optimization of a material, in this instance concrete. As depicted the System 7000 employs one or more AI/ML Algorithms 7110 (referred to as ROXI) which executes upon a computer system and accesses one or more Databases 7170 that store information acquired from a number of sources. These sources may include, but not be limited to, the Concrete Plant 7120 where data relating to aspects of the Concrete Plant 7120 is acquired and pushed or pulled to the Databases 7170. This may include, for example, data relating to aggregate moisture and grading, cement performance, material density, material temperature and raw materials and other data such as described with respect to FIGS. 7A and 7B, for example. The Databases 7170 also contain data acquired from Batch-Delivery 7130 relating to the batching and delivery of concrete from the Concrete Plant 7120 such as batch data across multiple batching plants, dispatch data, order tracking, GPS identifiers of trucks (and GPS data) as well as load status of delivery trucks leaving/returning to the Concrete Plant 7120.

Further, the Databases 7170 contain data from Truck Systems 7140 such as GPS data with time (for route tracking, time tracking, time stamping etc.), data from water and/or admixture dispensing systems on the trucks, and data relating to the concrete within the drum of the truck such as slump (as obtained by the embodiments of the invention described below), air content and water content.

The Databases 7170 also acquire Third Party Data 7150 using application programming interfaces (APIs) such as weather forecasts, weather stations (for actual weather data), and traffic data (either for dynamic routing/mix adjustment or analysis of deliveries etc.). Further an API may communicate with one or more laboratories performing tests, analysis, evaluations etc. upon concrete samples, raw materials, etc.

The Databases 7170 also acquire data from Construction Jobsites 7160 which may include, but not be limited to, data from embedded sensors such as described within this specification and others for temperature, strength, ion content, moisture etc. as well as data relating to pour time, pour location, framing, re-bar layout, etc.

Accordingly, the Databases 7170 comprise data acquired through the supply chain life cycle from delivered raw materials to the Concrete Plant 7120, production of the concrete (via Batch-Delivery 7130 and Concrete Plant 7120 for example), delivery information (via Batch-Delivery 7130 and Truck Systems 7140 for example), Third Party Data 7150, and deployment (via Construction Jobsites 7160 which includes embedded sensors).

As a result the AI/ML Algorithms 7110 can analyse data to provide data to one or more aspects of the system directly or to be employed in adjusting one or more systems. For example, the AI/ML Algorithms 7110 may provide continuous optimization of the concrete mixes produced by Concrete Plant 7210 and Batching System 7130, adjustments for raw material acceptance criteria, and timing data to Batching System 7130 for scheduling deliveries.

The AI/ML Algorithms 7110 may also provide adjustments to thresholds for dispensing systems, such as for water or admixtures, forming part of Truck Systems 7140, or where real time concrete data is accessible from the Truck Systems 7140 (either forming part of the truck directly or communicating with embedded sensors within the wet concrete) for real time dynamic adjustment and/or decision making.

The AI/ML Algorithms 7110 may also dynamically provide delivery acceptance at Construction Jobsite 7160 for a delivery or it may adjust the deployment of a delivery, e.g. a delivery scheduled for a support pillar is now more suited to pouring a floor at the same jobsite or another jobsite.

Within another embodiment of the invention the AI/ML Algorithms 7110 may in conjunction with data from Truck Systems 7140, and as also described below with respect to truck monitoring systems, determine that the remaining concrete after a delivery at one jobsite can be directly transported to another jobsite.

Within another embodiment of the invention the AI/ML Algorithms 7110 may in conjunction with data from Truck Systems 7140, and as also described below with respect to truck monitoring systems, determine that the remaining concrete after a delivery at one jobsite may be combined with an additional batch of concrete loaded into the truck when the truck returns where the Concrete Plant 7120 and Batching System 7130 generate a batch either to an existing recipe or to a custom recipe established in dependence upon a target concrete batch comprising the new load and the returning load.

Within another embodiment of the invention the AI/ML Algorithms 7110 may, by virtue of knowing the volume of concrete and performance of the concrete dynamically offer the load to one or more Construction Jobsites 7160, building contractors, etc. For example, various entities seeking smaller volumes of concrete with increased flexibility of pouring time/location may establish their requirements within a database accessible to the AI/ML Algorithms 7110. Accordingly, the AI/ML Algorithms 7110 may establish a returning truck may have a specific volume of a mix and match this to one or more requirements within the database, communicate the current immediate availability to those posting the one or more requirements, and upon receipt of an acknowledgement within a defined period of time re-route the truck(s) with this remaining concrete to the other locations. Further, by virtue of tracking the real time properties of the concrete within the truck the AI/ML Algorithms 7110 may dynamically adjust those to whom the option of a delivery is provided and advise others that due to the delay in acknowledgement the mixture is now no longer available.

The AI/ML Algorithms 7110 by having an overall perspective of the supply chain for multiple Concrete Plants 7120, Batching Systems 7130, Construction Jobsites 7160 etc. provide one or more dashboards to a user indicating the performance of the supply chain, status of different elements of the supply chain, performance of different elements of the supply chain, current deliveries etc.

The AI/ML Algorithms 7110 may also, based upon knowledge of the projected requirements, provided by an order fulfilment system (not depicted for clarity), projected traffic, projected weather etc. may establish appropriate schedules for the Concrete Plant 7120 and Batching System 7130. Where the AI/ML Algorithms 7110 are performing these functions for multiple Concrete Plants 7120 and Batching Systems 7130 then the AI/ML Algorithms 7110 may schedule concrete production and delivery to optimize aspects of the supply chain, such as limiting the number of changes to mixes made by a plant, adjusting delivery schedules, truck routing etc.

The AI/ML Algorithms 7110 may also trigger adjustments to raw material specifications, raw material delivery schedules or even raw material delivery locations. For example, assessment of sand being shipped may be more appropriate to mixes being scheduled at one Concrete Plant 7120 and/or Batching System 7130 than another, and hence the deliveries may be routed according to projected mixes and raw material quality assessments.

Optionally, a variant process may be implemented such as depicted in FIG. 8 wherein analysis is performed in respect of transportation of the construction material, e.g. concrete mix. In many concrete mix designs and deployments one or more admixtures are added to the concrete. These may be added at various points including, but not limited to, concrete batching, in truck, during deployment, and after deployment. Accordingly, FIG. 8 depicts an exemplary process for assessing admixtures, water etc. both in terms of which to add to the construction material based upon acquired historical data relating to their addition, delivery, performance etc. also determine when to add a particular admixture to a construction material batch and the quantity to add. For example, the analysis may determine that an admixture improving the low temperature pouring characteristics and initial curing of concrete is best added thirty minutes prior to pouring. Further, as this may be problematic for some or all deliveries the admixture(s) may be preloaded into one or more dispensers which are automatically triggered based upon downloading of a program to the concrete truck from the database for a specific delivery batch. In this manner, the admixture(s) are automatically added rather than when the truck driver can stop and add them. Equally, such analysis may determine that a batch having been loaded for two hours reaches a point where subsequent deployment will result in reduced performance or that the current projected environmental conditions will require all loads to be poured within a predetermined period of time if the concrete is required as a single contiguous block rather than multiple layers as a second pour is made upon a curing previous pour etc.

Accordingly, the exemplary process flow comprises first to seventh blocks 810 to 870 respectively, these being:
First block 810 wherein data acquired from in-truck and in-concrete sensors such as described above is collected and stored within the one or more remote servers storing information relating to the sensors as well as that established from concrete batch manufacturing plants, sensors embedded within the infrastructure elements, semi-embedded sensors associate with infrastructure elements, etc.;
Second block 820 wherein data relating to the mix transported for which data exists at the various points such as batching, truck loading, pouring, curing, ongoing life cycle monitoring etc. are retrieved and associated with the in-truck and in-concrete sensor data;
Third block 830 wherein the fresh concrete properties such as temperature, slump, air content, setting time etc. are retrieved and associated with the data existing at the various points such as batching, truck loading, pouring, curing, ongoing life cycle monitoring etc. are retrieved and associated with the in-truck and in-concrete sensor data;
Fourth block 840 wherein a plurality of artificial intelligence (AI)/machine learning (ML) algorithms and/or processes are employed upon the data in conjunction with data from other sources such as weather conditions and weather projections extracted from fifth block 850;
Sixth block 860 wherein the analysed effects of the mix constituents on the fresh concrete properties are established against the fresh concrete properties; and
Seventh block 870 wherein optimizations of the mix design, admixture dosage and time, water additions etc. are established.

Each of the exemplary processes described and depicted above exploits the acquisition of data from embedded sensors within the infrastructure.

Accordingly, the specifications within a database such as that employed in respect of FIG. 6 are those provided by one or more producers. Accordingly, the database includes mixture performance characteristics which may include: flowability/slump, strength versus time, air content, permeability, chloride diffusivity characteristics, etc. This database works as the back-end of a website to which a purchaser logs in to access. Optionally, website may be associated with a subscription service provided by one or more of the concrete producers, a sensor manufacturer, an industry organization, a regulatory authority etc. The purchaser enters as described in steps 610 to 630 several characteristics of the concrete required (such as a target strength of x at y days). The characteristics are matched to the database and several mixtures are identified. Then as described within steps 640 to 665 the identified mixes are then employed to refine the selection(s) in dependence upon the environmental, geographical factors etc. Accordingly, a mix may be rejected as the projected environmental conditions preclude it whilst another mix may be preferred.

Whilst the process flow depicted in FIG. 6 simply terminates after the user selects to end it based upon their being provided either in step 675, with projected performance of the selected existing specification based upon the location, time, historical environmental data, historical performance data etc., or in step 680, with specification options based upon the target characteristics defined by the performance and/or specification items selected by the user being matched against the available construction material specifications based upon the location, time, historical environmental data, historical performance data etc., where the process flow forms part of an online marketplace then the user upon determining a mixture is provided with the means to communicate to a producer through one or more means, such as electronic mail, electronic messaging, phone, website etc. Within embodiments of the invention accessing the website and subsequent steps of purchasing etc. may be integrated into project planning tools, supply chain management tools etc.

As discussed above in respect of FIG. 6 in step 660 historical performance data from sensors is extracted for a selected specification or available specification. Further, as discussed above in FIG. 7A a process flow for optimizing a manufacturing specification for a construction material according to an embodiment of the invention exploiting machine learning and artificial intelligence is presented. Also, as discussed above in respect of FIG. 8 an exemplary process for assessing admixtures, water etc. both in terms of which to add to the construction material based upon acquired historical data relating to their addition, delivery, performance etc. is described as well as determining when to add a particular admixture to a construction material batch and the quantity to add.

Accordingly, with the increasing use and deployment of sensors a substantial volume of data is collected and is poorly utilized. Further, a large volume of data collected throughout the service-life of concrete, from batching until demolition, is poorly utilized in the process of concrete design and specification. It is generally understood that several factors can affect the performance of concrete, including, but not limited to, mixture constituents, mixture constituent proportions, cement chemical composition, raw materials source, ambient conditions, distance from plant to site/traffic conditions, etc. Most of these pieces of information are known to concrete producers, while the concrete performance is measured on-site using quality assurance/quality control (QA/QC) methods using embedded sensors or well-established test methods (such as the slump test to determine concrete flowability). Although concrete producers may perform simple statistical analyses to detect anomalies for instance, this data is not combined and analyzed in a streamlined manner in order to, for example, predict concrete strength from mixture composition and ambient conditions. Due to the number of variables in this system and due to the complexity of establishing models to provide such estimates, the method of designing concrete mixtures has been largely based on experience, i.e. what worked for the last similar project will work for this one. This generally leads to losses in materials, energy and time due to the overly conservative choices as well as failure to identify anomalies, issues etc.

Accordingly, the inventors have established that it would be beneficial to provide a platform which provides:
centralised acquisition and storage of data collected from concrete producers regarding concrete mixture composition, cement chemical composition, raw materials type and source;
centralised acquisition and storage of data collected from embedded sensors, such as strength and temperature;
centralised acquisition and storage of QA/QC data collected from in-transit concrete properties (such as air content, slump and fresh concrete temperature), tests done on-site (such as slump/flowability); and
centralised acquisition and storage of data collected such as GPS location of concrete truck, GPS location of plant and jobsite and ambient humidity and temperature conditions.

Accordingly, this data is analyzed using machine learning algorithms in order to predict one or more factors, including, but not limited to:
concrete performance (such as strength, flowability, etc.);
perform mix optimization (to meet QA/QC results at a lower cost);
detect anomalies; and
project concrete deterioration.

An exemplary system according to an embodiment of the invention is composed of several components that are used for data collection and processing. These components comprising:
sensors that are used for data-collection on site where the acquired data may include, but not be limited to, concrete temperature, concrete humidity, concrete strength and sensor location;
a cloud platform component that collects and combines the data from the sensors;
a cloud platform component that collects data from the user on the concrete mixture design and QA/QC results;
a cloud platform that collects data on the surrounding conditions such as ambient (current and forecasted) temperature and humidity as well as traffic and location;
machine learning algorithm(s) that combine all of the collected data and perform algorithm training on the dataset(s); and
a cloud platform that receives data from the user, inputs it into the machine learning algorithm and displays predictions of compressive strength, slump, air content, temperature, permeability and other concrete properties and/or QA/QC results as would be readily understood by a person having ordinary skill in the art.

The collected data is divided, for example, into independent and dependent variables. The dependent variables include, but are not limited to:
mixture constituents' volumetric proportions;
cement chemical composition;
sand/gravel type and mineralogy;
chemical admixtures type and content;
current and projected ambient humidity and temperature;
concrete production, transition and placement conditions on construction jobsite; and
cost.
The independent variables include, but are not limited to:
strength;
workability;
air content; and
permeability.

The acquired data is collected and fed into one or more artificial neural network algorithms that determines correlations between the dependent and independent variables, in a process termed algorithm training. After performing training, the one or more artificial neural network algorithms are able to determine the concrete performance (dependent variables) through a knowledge of the mixture characteristics and external conditions (independent variables). Whilst the inventors describe embodiments exploiting artificial neural networks it would be evident that other approaches may be employed including, but not be limited to, decision trees, random forests, support vector machines, etc.

Within embodiments of the invention the one or more artificial neural network algorithms may also determine a required mixture recipe (independent variables) to achieve some QA criteria (dependent variables).

Within embodiments of the invention the one or more artificial neural network algorithms may also allow for detection of anomalies in one or more aspects of the manufacturing, transport, deployment, use life cycle as well as provide guidance to the user on best practices for their mixes using the historical data.

Within embodiments of the invention described above in respect of FIGS. 6 to 8 different methodologies with respect to exploiting machine learning and artificial intelligence for:
verification and/or specification of a construction material manufacturing composition based upon acquired material performance data from previous deployments acquired using sensors according to embodiments of the invention;
optimizing a manufacturing specification for a construction material; and
optimizing a construction material during transportation.

Within other embodiments of the invention machine learning and artificial intelligence may be employed to adjust a construction material for cost reduction and/or waste reduction. As described above and below in respect of SMAKs according to embodiments of the invention may be deployed within a construction material wherein the data they acquire relating to the construction material which is subsequently extracted and/or transmitted to a remote cloud based database. Such a cloud database may also obtain other data such as concrete mixture proportions either directly through user inputs, transfers from other databases such as those associated with the construction material supplier for example, or data acquired from the embedded sensors which had the construction material data stored within them. This additional data may also include a target strength which may be similarly input by an end user, such as an engineer, designer or specifier associated with the project to which deployment of the construction material relates or by the construction material manufacturer. As described with respect to SMAKs and databases within embodiments of the invention the construction material strength may be established via a method such as the maturity method either by algorithms in execution upon the SMAKs themselves or the remote cloud infrastructure.

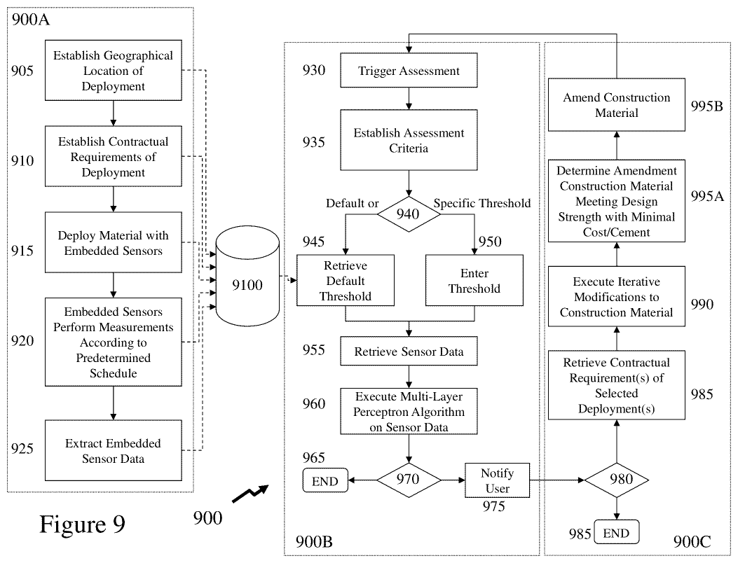

Accordingly, within an embodiment of the invention such as described herein or below with respect to FIG. 9 the construction material strength, predicted and/or actual, may be compared with the target strength. Where the actual strength is below the target strength by a predetermined percentage or amount then it has been described that an alarm or communication may be triggered indicating an issue with the construction material. However, where the actual strength differs from the target strength but is higher then such an alarm is not triggered as the construction material strength exceeds the design strength or target strength. However, within an embodiment of the invention where the actual strength exceeds the target strength by a predetermined percentage or amount then a user notification may be triggered to a user or users indicating, for example, “Potential exists for mixture optimization and cement savings.”

Accordingly, within an embodiment of the invention a machine learning algorithm or artificial learning algorithm may be employed, such as a multi-layer perceptron algorithm, which is trained on the historical strength data and construction material mixture proportion/composition data may be triggered/employed to calculate a percentage savings in cement, for example where the construction material is concrete, in order to reduce the construction material strength so that meets the specified target strength. Accordingly, a variance process is performed predicting strength for the same mixture proportions but for varying contents of the construction materials, e.g. cement for concrete. The content versus strength is used to calculate the percent decrease in the element of the content desired to allow the construction material to meet the design strength with minimal waste and/or cost. Accordingly, the variance analysis may assess those elements within the material composition having the highest cost. Subsequently, following these adjustments and realization of the savings, the strength is again monitored for further new cases of deployment of the construction material pouring, using the modified mixture, in order to compare the percentage saving and strength suggested by the algorithm with the actual savings achieved. This subsequent analysis may be employed for further algorithm training, e.g. further determination of weights in the multi-layer perceptron model where this is employed, allowing such suggestions to increase in accuracy and value as time proceeds.

Within embodiments of the invention this process may be executed prior to the construction material reaching its target strength using mathematical fitting of the temperature versus time data, for example, together with knowledge of the location and/or ambient weather forecast. Accordingly, embodiments of the invention exploit early predictions of concrete strength and therefore provide early suggestions of cement savings. For example, an initial concrete pour of foundations and/or lower levels of a building may provide sufficient data and early prediction that subsequent concrete pours employ adjust material compositions. Within another embodiment of the invention the target may be to achieve a minimum strength within a predetermined period of time in order to allow for increased speed of construction. Accordingly, the “cost” of a material composition may be established, for example, in terms of overall cost, material excess to that required to meet threshold performance (e.g. wasted cost/material), cost of project duration, etc.

Referring to FIG. 9 there is depicted an exemplary process flow 900 for optimizing a construction material for reduced cost/waste based upon embedded sensor data exploiting machine learning and artificial intelligence. As depicted the process flow 900 comprises a first sub-flow 900A, a second sub-flow 900B, and a third sub-flow 900C. Within first sub-flow 900A there are depicted first to fifth steps 905 to 925 relating to the deployment of SMAKs according to embodiments of the invention together with extraction of data from these SMAKs within a construction material. Accordingly, these steps comprise:

First step 905 wherein a geographical location of a deployment of SMAKs according to embodiments of the invention in conjunction with a construction material is established and communicated to a remove server 9100. This may be established by a designer, architect, owner of a structure being constructed, supplier of the construction material, contractor etc.

Second step 910 wherein contractual requirements of the deployment are established and communicated to the remote server 9100. Such requirements may include, but not be limited to, the target strength of the concrete, a minimum strength at a predetermined point in time, etc. This may be established by a designer, architect, owner of a structure being constructed, supplier of the construction material, contractor etc.

Third step 915 comprises deployment of the construction material with the embedded sensors wherein data relating to the deployment and/or SMAKs is communicated to the remote server 9100. This may be established by a supplier of the construction material, contractor etc. or it may be automatically acquired by data loggers, wireless interrogation, or wireless data acquisition for example.

Fourth step 920 wherein the embedded sensors perform measurements according to a predetermined schedule.

Fifth step 925 wherein the acquired data of the SMAKs and/or predicted material characteristics of a construction material is extracted. This data may be automatically acquired by data loggers, wireless interrogation, or wireless data acquisition for example and communicated to the remote server 9100.

Within second sub-flow 900B there are depicted first to tenth steps 930 to 975 respectively relating to the assessment of data acquired from SMAKs according to embodiments of the invention to determine whether an opportunity for optimization exists. Accordingly, these steps comprise:

First step 930 wherein an assessment process is triggered. This may be automatically triggered, for example based upon the remote server 9100 determining that SMAK data relating to a deployment for which contractual requirements exist has been acquired by the remote server 9100, or manually triggered by a user, e.g. a designer, an architect, a supplier of the construction material, etc.

Second step 935 wherein the assessment criteria are established, for example, target strength or strength at a predetermined time post-deployment from which the second sub-process 900B proceeds to third step 2940.

Third step 2940 determines whether a threshold between an achieved and/or predicted parameter versus a target for that parameter should be automatically established within the analysis of second sub-flow 900B or be established by a user. If the determination is automatic then the second sub-flow 900B proceeds to fourth step 945 wherein the second sub-flow 900B extracts the threshold from the remote server 9100 otherwise it proceeds to fifth step 950 wherein the user enters the threshold.

Sixth step 955 wherein the second sub-flow 900B extracts the SMAK data for the deployment or deployments that the user wishes to analyse. These deployment or deployments may, for example, be determined specifically by user entry of them or they may be determined based upon the user selecting one or more filters including, but not limited to, a geographical region of deployment, a type of construction, a construction material, a time of year, material supplier, and production location.

Seventh step 960 wherein the second sub-flow 900B executes a machine learning or artificial intelligence algorithm using the extract data in sixth step 955 together with the target material parameter(s), threshold etc.

In eighth step 970 the second sub-flow 900B determines whether there results of the analysis performed in sixth step 960 indicate an optimization is available or not. If not, the process proceeds to ninth step 965 and it and the process flow 900 end. If an optimization is available then the process proceeds to tenth step 975 and notifies one or more users.

Alternatively, within second step 935 rather than target strength or strength at a predetermined point in time other measurements may be employed including, for example, a specified slump versus actual measured slump or a specified air content versus actual measured air content. These values can be added to the application/sensor/server by user-input.”

Within third sub-flow 900C there are depicted first to sixth steps 980 to 995B respectively relating to the optimization of a construction material based upon a determination from the second sub-flow 900B indicates an optimization option exists. Accordingly, these steps comprise:

First step 980 wherein a user based upon the notification of the opportunity for an optimization of the construction material may decide whether to perform the optimization or not. If the user elects not to proceed the process proceeds to second step 985 wherein the third sub-flow 900C and process flow 900 end. If the user decides to perform an optimization then the process proceeds to third step 985.

Third step 990 wherein the third sub-flow 900C extracts the contractual requirement(s) of the deployment(s). These may be those employed in second sub-flow 900B or they may be those determined from a subsequent establishment process (not depicted).

Fourth step 990 wherein the third sub-flow 900C performs a process of iteratively assessing modifications to the construction material to determine resulting performance against the contractual requirements for the deployment or deployments being assessed.

Fifth step 995A determines whether any of the assessed modifications/amendments to the construction material meet the contractual requirements with minimal material.

Sixth step 995B wherein an amended construction material composition meeting the contractual requirements with minimal material is selected, either automatically or based upon user input. From sixth step 995B in third sub-flow 900C the process loops back to first step 930 of second sub-flow 900B.

The process loop feedback from sixth step 995B in third sub-flow 900C the process loops back to first step 930 of second sub-flow 900B results in the process flow 900 performing an ongoing construction material optimization. Whilst the embodiment of the invention described within process flow 900 relates to instances of the construction material parameter(s) exceeding the target specifications it would be evident that additional steps (not depicted for clarity) may be included which upon determination that the construction material parameter(s) do not exceed the target specifications such that the user or alternate user(s) are notified of the failure to meet target specifications. A failure may equally trigger a variant of third sub-flow 900C wherein the construction material composition is assessed for variations which will result in the construction material exceeding the target specifications.

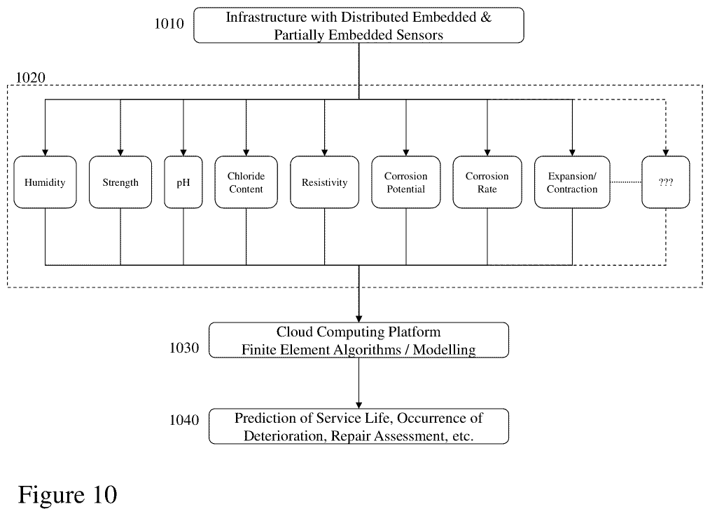

Each of the exemplary processes described and depicted above exploits the acquisition of data from embedded sensors within the infrastructure. As depicted in FIG. 10 an item of infrastructure has a plurality of embedded and non-embedded sensors associated with it as indicated in first block 1010. These measurements as indicated in second block 1020 may include, but are not limited to, humidity, strength, pH, chloride content, resistivity, corrosion potential, corrosion rate, and expansion/contraction. This data is then embedded to a cloud computing platform exploiting finite element algorithms and finite element modelling in third block 1030 wherein in fourth block 1040 the cloud computing platform generates data relating to prediction of service life, occurrence of deterioration, repair assessment etc.

As noted above with respect to the description of AI/ML processes, such as AI/ML Algorithms 7110 in FIG. 7C, these AI/ML processes may incorporate data acquired from one or more raw material monitoring systems and provide for discrete or continuous adjustments to mixes and/or batches. Different systems, methods and processes exist within the prior art to quantify and/or monitor raw material properties. These may include, but not be limited to, the moisture content of aggregate(s) and/or sand, the temperature of aggregate(s) and/or sand, moisture content of cement, the temperature of cement, water content of cement, the density of raw materials, the density of recycled/re-claimed water, aggregate(s) and/or sand particle size distribution, aggregate(s) and/or sand gradation, sand impurities, and cement impurities. The absolute values of these parameters and the subsequent the variability in these for the raw materials highly influence concrete properties. Accordingly, due to a lack of visibility producers may have on these changes, concrete mixes are typically over-designed and/or over-cemented in order to allow for or correct for these variations.

However, as noted above, the inventors AI/ML processes, such as offered via Giatec Scientific Inc.'s “SmartMix” platform, enables concrete producers to use these raw material measurements from these systems. The AI/ML processes combining within embodiments of the invention a portion exploiting AI/ML processes, a further portion employing defined logic and another portion employing defined algorithms. Accordingly, the AI/ML processes can identify the influence of changes in raw materials upon the concrete mix performance. The AI/ML processes further allow for dynamic and ongoing adjustment of mixes based upon raw material assessments of the raw materials delivered to concrete plants and used by batching systems.

For example, the inventors present below an exemplary use-case based upon the determination of temperature(s) of the raw materials, along with the mix design, in order to determine the temperature of the mixture. Accordingly, the AI/ML processes being provided with data relating to the temperature of a concrete mixture together with delivery information such as delivery distance and/or time to pour from truck loading, can determine whether admixtures should be added to maintain a consistent performance and if so, what admixture(s) and/or quantities etc. For example, for long delivery distances/times during hot weather, a retarder may be added to deliver the required and consistent slump at the point of delivery. The opposite may be required for short distances and/or cold weather where an accelerator may be added to maintain a reasonable setting time as specified by engineers and contractors for example.

An arbitrary example of the result from the AI/ML processes is depicted in Table 1 below. For example, the AI/ML processes based upon historical data for concrete production, delivery time/distance, ambient temperature, and concrete mix temperature formulates the required admixture(s) and volumes. Whilst, the exemplary example in Table 1 is presented as a table (which may for example be used where automated variable admixture dispensing systems are not available where a dispatcher, truck driver or other operative adds the admixtures as defined, e.g. using pre-measured volumes. Table 2 defining a finer granularity where a dispensing system is triggered allowing for the finer control and removal of operator error.

However, within other embodiments the AI/ML processes may render a function which is provided to a dispensing system or the AI/ML processes may directly trigger the dispensing system. In these latter examples operators may be removed through automation of the dispensing process where the system now provides but more importantly the admixture dispensing may be continuously adjusted to reflect subsequent variations in ambient temperature, slower truck speed, faster truck speed, traffic issues, truck re-routing etc.

TABLE 1
Exemplary Additive Table for Operator Based Admixture Addition

| | Distance | | | | |
|---|---|---|---|---|---|
| Temperature | 10 km | 15 km | 20 km | 25 km | 30 km |
| 5° C. | Add 2x | | Add 1x | | |
| | Accel- | | Accel- | | |
| | erator | | erator | | |
| 10° C. | | Add 1x | | | |
| | | Accel- | | | |
| | | erator | | | |
| 20° C. | | | | Add 1x | |
| | | | | Stabilizer | |
| 30° C. | | | | | Add 3x |
| | | | | | Retarder |

TABLE 2
Exemplary Additive Table for Operator Based
Admixture Addition (% of Concrete Weight)

| | Distance | | | | |
|---|---|---|---|---|---|
| Temperature | 10 km | 15 km | 20 km | 25 km | 30 km |
| 5° C. | 2% | 1.5% | 1% | 0.5% | — |
| | Accel- | Accel- | Accel- | Accel- | |
| | erator | erator | erator | erator | |
| 10° C. | 1.5% | 1% | 0.5% | 0.25% | — |
| 20° C. | — | 0.6% | 0.5% | 1% | 1.25% |
| | | Stabilizer | Stabilizer | Stabilizer | Stabilizer |
| 30° C. | 0.5% | 1% | 1.5% | 2.1% | 3% |
| | Retarder | Retarder | Retarder | Retarder | Retarder |

According to another embodiment of the invention sensors may be used to determine the density of water associated with one or more sources used within the mixture of a batch, these may be, for example, direct mains water feed, rest water within a tank, recycled water, or reclaimed water. From this density it being possible to determine the total solids content. For example, water from a mains feed may have low solids content but water from washing a truck drum out may have high water solids initially unless it sits for an extended period of time and some settles out.

These measurements may simply be used for compliance and to ensure that the material delivered meets the specification. These measurements may be performed for example using an optical light absorption sensor, an ultrasonic sensor, or any other concept used to determine fluid density or total suspended solids. The data from the sensor(s) can be used to determine the total solids, which can be subsequently used to adjust the amount of cement and fine aggregates used in the mixture. Using the example above the volume of fine aggregates may be reduced if the water being used was recently used to wash out a truck drum versus from a direct mains feed.

Accordingly, in this manner the producer is able to reduce the quantities of fine aggregates, fresh water and cementitious materials all at once, by utilizing sensor measurements. The adjustments may be automated, for example via AI/ML processes, or defined by a user logic where they can define the amount of cement and fine aggregate to be subtracted from the mixture for a certain amount found in rest water for the volume of rest water employed. Obviously, automated methodologies reduce human error and allow the mixture being produced to have varying quantities of water from different sources automatically corrected for,

Within another embodiment of the invention measurements on aggregate(s) within the aggregate supply, for example aggregate moisture (which may be measured through a microwave time-domain reflectometry or near-infrared sensors for example) or aggregate particle size distribution (measured using shadowgraphy or image analysis for example) can be used in conjunction with AI/ML processes to adjust mixtures on-the-fly where data from these sensors can be combined with user-defined logic (for instance specific particle size distribution for aggregate blends) to adjust mixes to ensure consistency regardless of aggregate variations. The data can also be used to adjust moisture content (significant prior art) to maintain total water content regardless of moisture content variations. If the measurements are performed as material is employed then the AI/ML processes can adjust in real time rather than using data from a batch of material delivered as this may vary significantly given the volumes of material employed in some concrete plants/batching operations.

In all of the aforementioned concepts, the adjustments may occur based on AI/ML processes, for example, discretely or in combination with user-defined logic or algorithms. AI/ML processes offer enhanced performance as they learn from and adapt using historical data of material variations versus final material property(ies) variation(s) and can react to real time data as well. For example, an AI/ML process may define the aggregate portion for a mixture based upon data relating to the aggregates, cement, water etc. However, if for example it is producing sufficient concrete for 100 trucks then it can not only adjust for the raw material feed but also from data relating to the concrete during transit and the deployments from these truck loads.

In order to simplify data collection and combine both of sensor data and adjustment logic, it is desirable to collect this data remotely without operator intervention in order to perform on-the-fly changes to mixtures using AI/ML processes. Accordingly, the inventors have also worked to implement wireless communication capabilities to the wide range of sensors which are traditionally connected to an on-premises system. In this manner, wireless communications can be made directly to the Databases 7170 in FIG. 7 for example through hubs, micro-hubs etc. rather than relying upon a central collection/transmission from the on-premises system. This also allows for additional sensors to be added and feed data to the databases of the AI/ML processes and/or reconfigured without consideration of architecture to the on-premises system. Further, a producer can leverage data from all of their plants discretely or in conjunction with one or more anonymised databases.

For example, multiple producers may provide data to the databases of the AI/ML processes. In some embodiments the data fed may be absent identifying information such as producer name, plant identity, truck identity, GPS data etc. However, within other embodiments of the invention the multiple producers knowing that the AI/ML processes improve based upon the volume of data analysed may provide everything to the database where the AI/ML processes by virtue of being hosted by a third party access the data and provide recommendations, mixture data etc. to the producers without the source raw data for the AI/ML processes being accessible to other producers than the producer providing it. A producer may thereby leverage a significantly larger volume of data either regionally, nationally or internationally. For example, short spikes in ambient temperature >35° C. in Canada may mean the data accessible to a producer in Canada may be limited. However, if the AI/ML processes can access data from the Middle East, southern United States etc. allows then the volume of data is significantly increased.

### Slump Monitoring

### Accelerometer Based Non-Invasive Slump Monitoring

A method of monitoring concrete slump and adjusting the concrete during transit and delivery would provide significant benefits to concrete producers in terms of reducing concrete load rejection, reducing disposal of unused/rejected concrete, reducing use of water/admixture adjustments, reducing cementitious materials and subsequently significant cost savings. Reductions in concrete load rejection would also benefit those to users of the concrete by reducing production delays during concrete pours or reworking of poured concrete to correct for material quality issues.

Within the prior art two methods have been generally employed to monitor slump in transit. The first method relies upon measuring the rotation and torque applied to the mixing drum from which the Bingham parameters (primarily yield stress) of the mixture can be determined. Such a method being described within U.S. Pat. No. 8,020,431 “Method and System for Calculating and Reporting Slump in Delivery Vehicles.” Concrete is a non-Newtonian fluid whose rheological properties are represented by a Bingham model where parameters that represent the flow properties of the material are τ₀ and μ where the flow equation of concrete is defined in the Bingham model by where μ is slope of rate of shear rate (γ) with shear stress (μ) and τ₀ the minimum stress value below which the concrete will not flow.

The other method relies upon a probe which is installed on the inner wall of a drum of a concrete delivery truck where deformation of the probe as the drum rotates is monitored and used to calculate a resistance pressure. From this resistance pressure the Bingham parameters may be deduced. This being described within U.S. Pat. No. 9,199,391 “Probe and Method for Obtaining Rheological Property Value” for example.

However, these aforementioned methods are invasive, costly, require significant adjustments to the drum/truck system to operate and require significant maintenance. Previously, within PCT/CA2019/000057 “Construction Material Assessment Method and Systems” some of the inventors established a technique employing a sensor monitoring electrical resistivity wherein this exhibits a time dependent response where the electrical resistivity is high when the sensor is in air and low when the sensor is within the concrete material. Accordingly, based upon the frequency, amplitude, phase shift (between the resistivity fluctuation cycles and the drum revolution cycles), and duty cycle of the electrical resistivity measurement the inventors established yield stress, plastic viscosity and slump for the concrete within the drum. Whilst non-invasive the method still requires a sensor inside the drum.

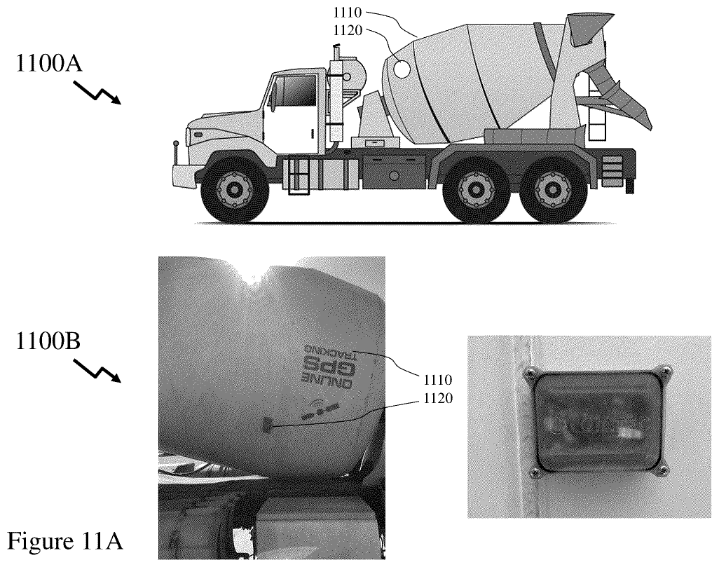

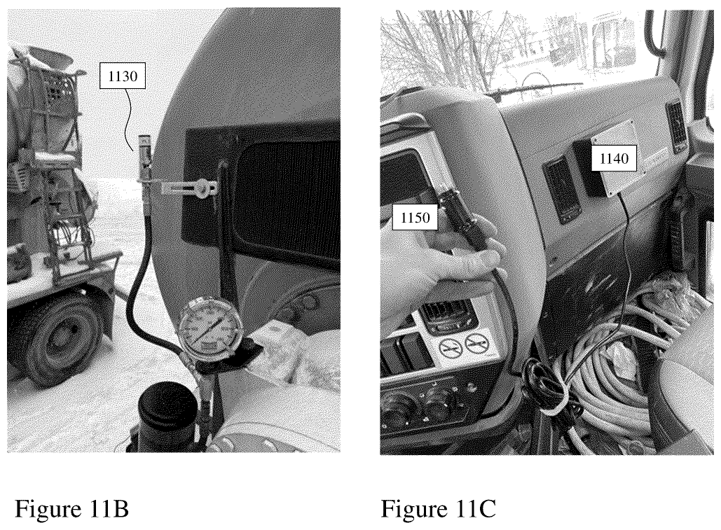

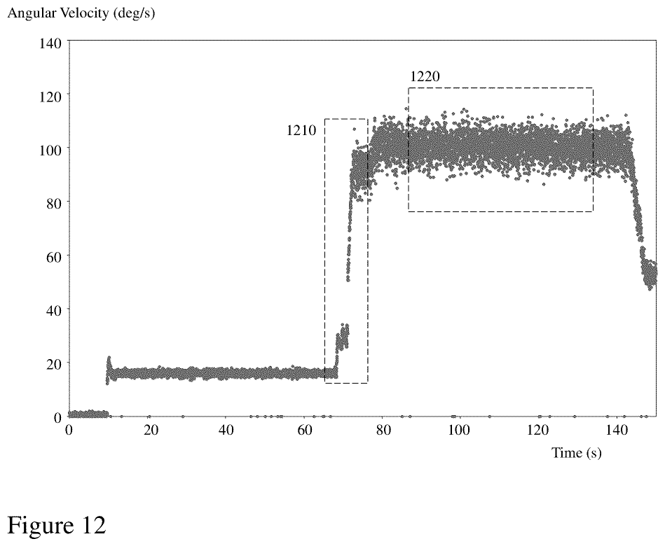

However, the inventors wished to establish a method wherein the slump can be monitored non-invasively without accessing the drum internally or the drum systems, including the engine. The benefits being significantly reductions in the cost of installation and maintenance of such a system. Accordingly, the inventors established a method, as depicted in FIG. 11 in Schematic 1100A and Installation 1100B, wherein a Sensor Module 1120 is disposed upon the exterior surface of the Drum 1110. The method established by the inventors monitors the acceleration and angular velocity of the Drum 1110 through an externally installed gyroscopic sensor within the Sensor Module 1120 which determines the acceleration, angular velocity and orientation of the drum. For example, the Sensor Module 1120 may be permanently attached to the Drum 1110 or it may be demountably attached to the Drum 1110. For example, the Sensor Module 1120 may be magnetically attached to the Drum 1110. Whilst the Sensor Module 1120 has been depicted upon the exterior of the Drum 1110 it would be evident that within other embodiments of the invention the Sensor Module 1120

Accordingly, a system comprising a multi-axis accelerometer sensor provides data that is converted by a microprocessor to changes in angular velocity (i.e. changes in revolutions per minute (RPM) of the Drum 1110). These changes in the Drum 1110 may, for example, be forced by the operator of a system (e.g. truck) associated with the Drum 1110 or caused by agitation of the mixture within the Drum 1110. First Region 1210 depicts the angular velocity during a change in rotation of the Drum 1110 whilst second Region 1220 depicts the angular velocity detected during constant drum agitation at a constant RPM.

The system generates in dependence upon measured angular velocity the torque exerted by the engine driving the Drum 1110. The time required to establish a change in RPM or angular velocity of the drum is related to the torque applied on the drum, the drum's diameter, the weight of the drum and the properties of the concrete including its weight. Further, by determining the engine's torque at several RPM levels, the yield stress of the Bingham fluid can be determined and used to calculate the concrete slump through defined correlations. The relationship between the torque at different RPMs is used to calculate yield stress by extrapolating the relationship to the torque required at 0 RPM.

For example, during routine rotation of the drum during transport a controller may cycle the drum through two or more rotation rates automatically. Based upon the sensor measurements the system can then automatically determine the yield stress etc. This data can then be pushed to a remote server for storage and/or processing such as described within this specification or employed to provide a driver or other operator/user associated with the drum data relating to the condition of the concrete. Optionally, the overall system may only trigger communications to a user/operator etc. based upon determining that a property of the concrete has gone without predetermined boundary conditions thereby requiring that an action be taken, e.g. addition of water to the concrete mixture, addition of an additive to the concrete mixture, termination of the delivery etc.

The inventors have extended the system described above to a three-component system which in addition to determining slump also allows concurrent determination of load size during delivery of concrete in a ready-mix concrete truck. The system contains:
a Sensor Module 1120 installed on the outside of the drum either permanently attached or demountable attached (e.g. magnetically fixed) which monitors angular velocity, positioning and acceleration/tilt;
a Hydraulic Sensor to monitor the drum's hydraulic pressure; and
a Data Acquisition (DAQ) system installed upon the truck, for example within the truck cabin.

An example of the Hydraulic Sensor being depicted in FIG. 11B where the Hydraulic Sensor 1130 is coupled to the hydraulic system of the drum. An example of the DAQ being depicted by DAQ 1140 in FIG. 11C where the DAQ 1140 connects to the truck power through an interface such as the Connector 1150 depicted.

This system of Sensor Module 1120, Hydraulic Sensor 1130 and DAQ 1140 provides for dynamic measurement of the concrete's slump within the drum. The method is based upon monitoring the hydraulic pressure of the drum before and after concrete introduction (i.e. before and after concrete batching). This difference in pressure, related to the introduced concrete, is monitored over a range of angular velocities. Accordingly, from the angular velocity and the hydraulic pressure the shear rate and shear stress applied to the concrete can be monitored.

### What Part of the System is Hydraulic/What Hydraulic Pressure is Monitored?

For example the measurement data is applied to one or more models, which may include for example a Herschel-Bulkley model or Bingham model, depending upon the concrete constituents, to determine the yield stress, viscosity/consistency and flow index of the concrete. The yield stress is then used to determine the slump of the concrete under consideration.

Accordingly, the inventors system allows the determination of slump in a manner that is non-intrusive, can be performed during transportation of the concrete or upon the concrete at the delivery location prior to pouring. This provides significant advantages compared to prior art methods that require significant intrusion, installation and maintenance. Data from this system can be used along with the concepts defined within this specification to exploit AI/ML processes that allow for processes, models and algorithms to be established and dynamically employed for adjusting mixes based on raw material variation

The inventors also have established that measurements of hydraulic pressure and angular acceleration can be used to determine load size (or the center of mass) of the system under consideration. Knowing that the net torque acting on concrete drum is directly proportional to the angular acceleration and moment of inertia/center of mass of the concrete mass then by monitoring changes in hydraulic pressure and the associated changes in angular velocity (i.e. angular acceleration) within an assumed system efficiency the concrete load size can be determined. Whilst the system efficiency for improved accuracy should be considered as truck dependent it can be deduced from measurements taken of the empty truck and hence can be defined per truck.

Accordingly, the hydraulic pressure acting on the drum is monitored using a pressure gauge, e.g. Hydraulic Sensor 1130, and the acceleration of the system is monitored, e.g. using a Sensor Module 1120. The data from both systems are acquired by the data acquisition system, DAQ 1140. The changes in acceleration and changes in pressure are correlated to yield the center of mass/moment of inertia/load size of concrete within the concrete drum. This may be performed by the DAQ 1140 or remotely.

### Would be Good if had Equation(s)

Accordingly, this system allows concrete producers to determine the volume of concrete not only initially loaded into the truck, to ensure compliance with vehicle regulatory requirements on maximum weight, for example, it also allows for the volume of concrete being rejected or returned to a concrete plant after the truck has reached the delivery location to be determined along with the slump of the concrete being returned. This, as noted above, allows AI/ML processes to either adjust the returned concrete to meet specific criteria for a different concrete at the same jobsite or another jobsite, re-direct the rejected concrete to a different jobsite or different pour at the same jobsite to avoid waste and increase material utilization, or configure an additional mixture to add to the returned material to meet other specific criteria of a further pour required at the jobsite or a further jobsite.

### Differential Pressure Based Slump Monitoring

On-site slump measurements are time-consuming, costly and have relatively poor repeatability and they are prone to errors. Such tests are typically carried out using a metal mould in the shape of a conical frustum known as a slump cone or Abrams cone unless the concrete is too fluid. Examples of such tests include the “Standard Test Method for Slump of Hydraulic-Cement Concrete” (US Standards ASTM C 143 or AASHTO T 119) “Testing Fresh Concrete-Part 2: Slump Test” (European Standard EN 12350-2).

Accordingly, it would be desirable to provide a method for monitoring delivered concrete loads onsite which is rapid, cost-effective, and removes errors from working practices and/or interpretations. The inventors have developed methods and systems based upon the determination of concrete's lateral pressure and correlations of these pressure measurements to the concrete's rheological characteristics.

Accordingly, the methods and systems may exploit a probe with a diaphragm-type pressure sensor that is used to measure the pressure exerted by the concrete on the probe when embedded in concrete. Flowable concrete exerts more lateral pressure on the probe's sensor than concrete with lower flowability (i.e. low slump concrete).

Optionally, the probe and/or sensor may be fitted with or employed in conjunction with a haptic engine such as an eccentric rotating mass motors (ERMs) or linear resonant actuators (LRAs) for example. The haptic engine will vibrate the probe wherein the sensor detects changes in pressure or another sensor detects acceleration of the probe within the concrete mixture. The force required to maintain the vibration at different oscillation frequencies can be used to determine the concrete's rheological properties, i.e. yield stress, by forcing vibrations at varying frequencies and sensing pressure.

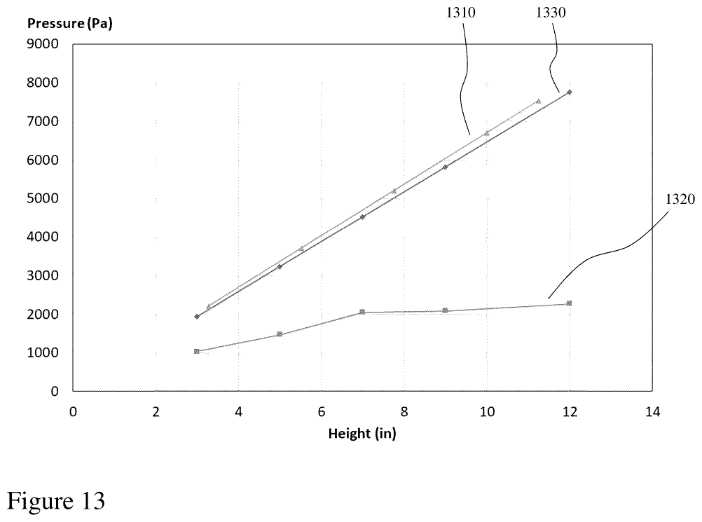

Accordingly, a probe according to an embodiment of the invention for determining differential pressure measurements to establish concrete density on-site/upon delivery may comprise a pair of diaphragm-type pressure sensors which are installed at a predetermined spacing. Accordingly, based upon the measurements of the pressure the concrete differential pressure can be determined and the concrete density calculated. Referring to FIG. 13 there are depicted first to third Plots 1310 to 1330 respectively depicting the different pressure as a function of vertical spacing between the pair of diaphragm-type pressure sensors. First Plot 1310 depicts the pressure differential versus spacing for a first concrete mixture whilst second Plot 1320 depicts the pressure differential versus spacing for a second concrete mixture. Third Plot 1330 depicts the pressure differential versus spacing for a hydrostatic equivalent fluid.

As inclination of the probe would affect the measurements, i.e. adjusting the vertical spacing between the pair of diaphragm-type pressure sensors within the concrete, the probe contains a sensor (inclinometer) providing for measurement of the probes inclination. Accordingly, the data from the sensor (inclinometer) is employed to correct for the inclination angle such that the actual vertical distance between the pair of diaphragm-type pressure sensors is employed rather than the nominal design value for the probe in a vertical position.

Accordingly, the established differential pressure corrected for the actual vertical distance between the pair of diaphragm-type pressure sensors can be employed to provide a determination of the concrete at a work site with ease, flexibility, and removing errors arising from workers etc.

The probe may be a discrete self-contained probe with a wireless connection to a monitoring device, e.g. a PED with a concrete property software application in execution upon it or a PED pushing the data to remote storage for processing by a concrete property software application.

The probe may be a discrete self-contained probe with a wired connection to a monitoring device, e.g. a PED with a concrete property software application in execution upon it or a PED pushing the data to remote storage for processing by a concrete property software application.

Optionally, the probe may provide a visual indication of the pressure differential or flowability of the concrete to a user (e.g. via a display, colour coded bar display etc.).

Optionally, the probe may provide an audible indication of the pressure differential or flowability of the concrete to a user.

For example the inclinometer may employ a single axis microelectromechanical system (MEMS) inclinometer or a dual axis MEMS inclinometer. An advantage of a dual axis MEMS inclinometer being simultaneous measurement of two-dimensional (X-Y plane) tilt angles, digital compensation and calibration for non-linearity, for example for operating temperature variation, resulting in higher accuracy over a wider measurement range, and accelerometer sensors may generate numerical data in the form of vibration profiles to enable tracking and assessment of probe stability and/or alignment in real-time and verify positional stability.

Optionally, an inclinometer with gyroscope may be employed. As an inclinometer measures the angle of an object with respect to the force of gravity, external accelerations like motion, vibration or shock introduce errors in the tilt measurements. These can be overcome through the user of a gyroscope in addition to an accelerometer.

Optionally, diaphragm-type pressure sensors may be deployed within a concrete dispensing system such as a boom pipe or hose of a concrete boom truck (also known as a concrete pump truck) or piping associated with a pumping system moving concrete from delivery trucks to the location of its deployment. In such instances a combination of at least a pair of diaphragm-type pressure sensors and an inclinometer would allow the flowability of the concrete etc. to be determined in real time as the concrete is deployed. Optionally, the pump pumping the concrete may be programmed to periodically pause allowing the pressure of a column of stationary concrete to be measured and the flowability determined.

Optionally, diaphragm-type pressure sensors may be deployed within a concrete dispensing system in conjunction with a velocity sensor or velocity sensors without pausing the flow of concrete wherein the pressure differential s adjusted in dependence upon the velocity such that the flowability may be determined upon flowing concrete. Accordingly, the system may be calibrated to measure the pressure dynamically during pumping as the concrete flows rather than during a pause in pumping.

Optionally, flowability may be determined from static and dynamic pressure measurements.

Optionally, for fixed pump characteristics and known pipe cross-section a velocity of concrete flow may also be determined from a sensor or sensors to provide an alternate measure of flowability.

Within such systems the requirement is that the vertical separation between the pressure sensors is known from which the pressure measurements are made. Accordingly, the

### Mixture Optimization Through Thermal Modelling

As outlined above machine learning (ML) and/or artificial intelligence (AI) based systems may be employed in order to define, specify and/or optimize concrete mixtures being delivered for mass concrete applications (e.g. dams, foundations, wind turbine footings, etc.). In such systems it would be desirable to employ a model for the prediction of concrete temperature rise and heat development. In order to do so, the factors influencing concrete heat generation during concrete hydration (the reaction between water and cement) were collected by the inventors. These factors include the specific heat and thermal conductivity of concrete proportions (e.g. cement, water and aggregates), the thermal capacity of concrete forms, ambient temperature, solar absorption, cementitious materials chemistry and fineness, chemical admixtures characteristics, etc.

The inventors established an initial model to simulate the degree of hydration development over time for concrete. This model was compared to literature data on reported degree of hydration and was found to mimic these experimental data closely. This initial model was then extended as a heat transfer model to simulate the heat generated during concrete hydration and the heat dissipation/gain due to ambient conditions.

The developed heat transfer finite-difference model was then compared to literature data from reported experimental heat rise measurements in order to evaluate its accuracy and shortcomings. The model was found to perform well in cases where the boundary conditions (e.g., formwork characteristics) were known. In cases where such boundary conditions were not clearly stated in the literature these factors led to a reduction in the accuracy of the model as is common with most modelling when boundary conditions become e model tended to perform poorly. Accordingly, the inventors in establishing a software application for use by construction material producers, formwork manufacturers, architects, installers, etc. established from collected data a series of standard configurations and/or options based upon the most commonly used formwork materials (e.g., steel formwork) as part of the modelling suite to assist these end-users.

In order to validate the model three projects were modelled using the model established by the inventors. These three projects being those of a ready-mixed concrete producer partner in California wherein the simulations of concrete heat rise were performed and compared to measurements acquired using in-situ temperature sensors, namely Giatec Scientific's SmartRock™ and/or BlueRock™ MBEDSENS devices. In all instances the maximum deviation between the model and measured temperatures was 2.8° C. (5 F).

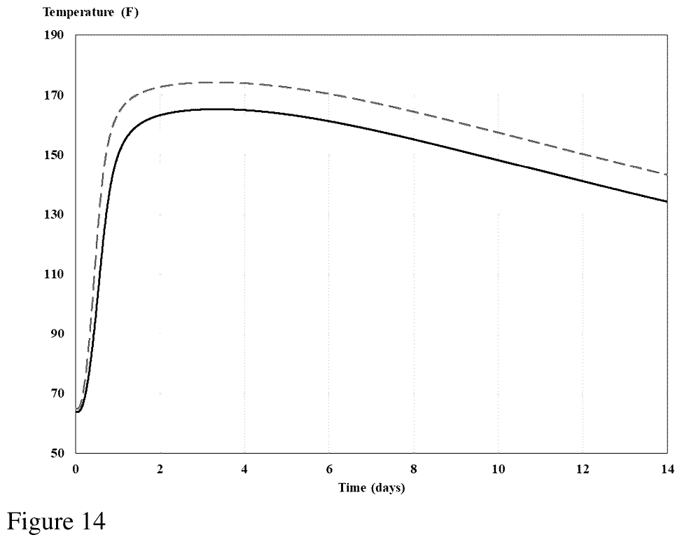

FIG. 14 depicts the modelled temperature from the model established by the inventors showing the increase in temperature for two different concrete mixes, Mix 1 1410 and Mix 2 1420, over a period of 14 days showing that the peak temperature exceeds 165 F (approximately 74° C.) and 174 F (approximately 79° C.) respectively.

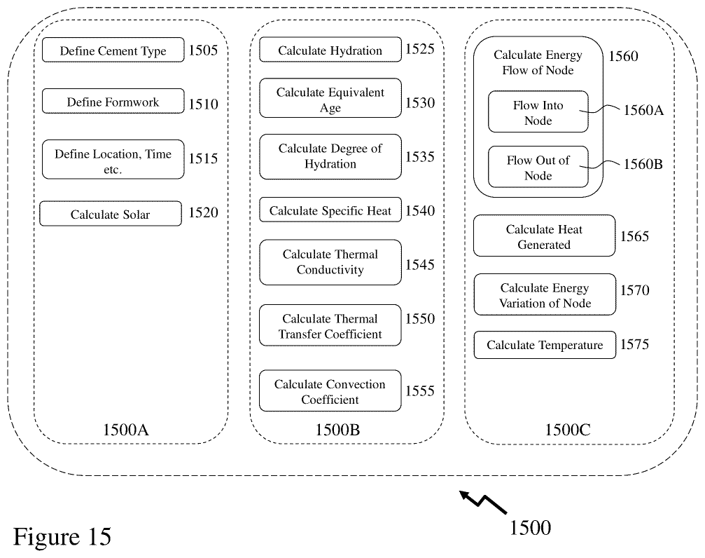

Referring to FIG. 15 there is depicted an exemplary Software Module Schematic 1500 for a software application executing a model according to an embodiment of the invention. As depicted the Software Module Schematic 1500 comprises first to third Module Blocks 1500A, 1550B and 1500C respectively. First Module Block 1500A depicts exemplary first to fourth Configuration Modules 1505 to 1520 relating to the definition of the structure being modelled. Second Module Block 1500B depicts exemplary first to seventh Definition Modules 1525 to 1555 respectively relating to establishing properties of the concrete. Third Module Block 1500C depicts exemplary first to fourth Modelling Modules 1560 to 1575 respectively.

Referring initially to first Configuration Module 1505 a user establishes a cement type composition for the model to execute the modelling with. This may, for example, be by selecting a standard composition from a menu or based upon the user establishing a new composition. Referring to Table 1 below there are depicted three exemplary compositions.

TABLE 1
Exemplary Cement Type Compositions

| Chemical | Type | Type | Type |
|---|---|---|---|
| Composition | I | II | V |
| C₃S | 0.583 | 0.635 | 0.4985 |
| C₂S | 0.147 | 0.162 | 0.2441 |
| C₃A | 0.11 | 0.073 | 0.018 |
| C₄AF | 0.061 | 0.094 | 0.161 |
| SO₃ | 0.03 | 0.027 | 0.027 |
| Free CaO | 0.008 | 0.007 | 0.008 |
| MgO | 0.013 | 0.015 | 0.008 |
| Na₂O | 0.003 | 0.002 | 0.0027 |
| Na₂Oeq | 0.00563 | 0.00529 | 0.00421 |
| Blaine | 377.1 | 354.3 | 410 |

Within second Configuration Module 1510 the user establishes the formwork by defining, for example, the shape of the formwork, material for the formwork, etc. Subsequently, within third Configuration Module 1515 the user defines a location for the deployment being simulated together with time data. Accordingly, the location and time data is employed to extract from one or more databases data relating to environmental conditions for the deployment such as ambient air temperature, ground temperature (if appropriate), and hours of sunlight. Further the location data is parsed to established latitude and longitude information if the location is specified in another format such as defining a location within a map, online map etc.

The location data is employed within fourth Configuration Module 1520 to establish solar irradiance upon the concrete structure being modeled together with direct and indirect uncertainties in the prediction of tilted irradiance for solar engineering applications. Accordingly, the fourth Configuration Module 1520 generates Equations (1) to (3) respectively. Equation (1) being modified (adapted) for Python (the high-level general-purpose programming language employed by the inventors) to Equation (4) where Eₛ is the solar energy absorbed by the exterior node

$$( \frac{W}{m \cdot K} ) ,$$

DNI is the Direct Normal irradiance

$$( \frac{W}{m \cdot K} ) ,$$

### DHI the Diffuse Horizontal Irradiance

$$( \frac{W}{m \cdot K} ) ,$$

### GHI the Global Horizontal Irradiance

$$( \frac{W}{m \cdot K} ) ,$$

s is the inclination of the surface (rad), sα is the surface solar absorptance, and cc is the cloud cover in decimals.

$${E}_{s} = {E}_{b n} \cdot cos ( ϑ ) + {E}_{d} \cdot {R}_{d} + ρ \cdot E \cdot {R}_{s} \qquad Equation ( 1 )$$

$$GHI = DHI + DNI \cdot cos ( Z ) \qquad Equation ( 2 )$$

$$R = {R}_{0} \cdot ( 1 - 0.75 \cdot {n}^{3.4} ) \qquad Equation ( 3 )$$

$${E}_{s} = DNI \cdot cos ( max ( - s + {cos}^{- 1} ( \frac{GHI - DHI}{DNI} ) , s - {cos}^{- 1} ( \frac{GHI - DHI}{DNI} ) ) ) + ( DHI + ρ \cdot GHI ) \cdot \frac{1 + cos ( s )}{2} \cdot sa \cdot ( 1 - 0.75 \cdot {cc}^{3.4} ) \qquad Equation ( 4 )$$

It would be evident that other Configuration Modules may be employed in addition to those described. The data established within first Module Block 1500A is employed within second Module Block 1500B relating to establishing properties of the concrete. Second Module Block 1500B comprises first to fifth Definition Modules 1525 to 1545 respectively.

First Definition Module 1525 calculates:
β=hydration slope parameter;
τ=hydration time parameter (h);
αᵤ=ultimate degree of hydration;
Hᵤ=total amount of heat generated at 100% hydration (J/kg); and
E_{α}=activation energy (J/mol).

These are given by Equations (5) to (10) respectively although the inventor modified versions allowing them to be executed within a Python based embodiment of the invention are defined by Equations (11) to (15) respectively where:

$$cem = cement content ( \frac{kg}{{m}^{3}} ) ;$$

$$SG = slag content ( \frac{k g}{{m}^{3}} ) ;$$

$$SF = silica fume content ( \frac{k g}{{m}^{3}} ) ;$$

$$FA = fly ash content ( \frac{k g}{{m}^{3}} ) ;$$

$${W}_{c m} = weight of cementitious materials ( \frac{k g}{{m}^{3}} ) ;$$

p_{c}_{₃}ₛ=proportion of alite in the cement;
p_{c}_{₂}ₛ=proportion of belite in the cement;
p_{c}_{₃}_{A}=proportion of aluminate in the cement;
p_{c}_{₄}_{AF}=proportion of ferrite in the cement;
pₛₒ_{₃}=proportion of total sulfate in the cement;
p_{FreeCA}=proportion of calcium oxide in the cement;
p_{Mgo}=proportion of magnesium oxide in the cement;
p_{Nα}_{₂}_{O}=proportion of sodium oxide in the cement;
p_{FA-CαO}=proportion of calcium oxide in the fly ash;

$${p}_{{Na}_{2} Oeq} = proportion of equivalent sodium alkalis in the cement = {p}_{{Na}_{2} O} + 0.658 \cdot {p}_{{K}_{2} O} ;$$

$$Blaine = cement Blaine fineness ( \frac{{m}^{2}}{kg} ) ;$$

ACCL=ASTM type C accelerator dose;
LRWR=ASTM Type A water reducer dose;
MRWR=mid-range water reducer dose;
NHRWR=Type F naphthalene HRWR dose;
PCHRWR=ASTM Type F polycarboxylate based high-range water reducer dose; and
WRRET=ASTM Type B & D water reducer/retarder dose

$$β = {e}^{( - 0.464 + 3.41 \cdot {p}_{{C}_{3} A} \cdot {p}_{cem} - 0.846 \cdot {p}_{slag} + 107 \cdot WRRET + 33.8 \cdot LRWR + 15.7 \cdot MRWR + 38.3 \cdot PCHRWR + 8.97 \cdot NHRWR )} \qquad Equation ( 5 )$$

$$τ = {e}^{( 2.92 - 0.757 \cdot {p}_{{C}_{3} S} \cdot {p}_{cem} + 98.8 \cdot {p}_{{Na}_{2} O} \cdot {p}_{cem} + 1.44 \cdot {p}_{slag} + 4.12 \cdot {p}_{FA} \cdot {p}_{FA - CaO} - 11.4 \cdot ACCL + 98.1 \cdot WRRET )} \qquad Equation ( 6 )$$

$${α}_{u} = \frac{1.031 \cdot w / cm}{0.194 + w / cm} + {e}^{( - 0.0885 - 13.7 \cdot {p}_{{C}_{4} AF} \cdot {p}_{cem} - 283 \cdot {p}_{{Na}_{2} Oeq} \cdot {p}_{cem} - 9.9 \cdot {p}_{FA} \cdot {p}_{FA - CaO} - 339 \cdot WRRET - 95.4 \cdot PCHRWR )} \qquad Equation ( 7 )$$

$${H}_{cem} = 500 \cdot {p}_{{C}_{2} S} + 260 \cdot {p}_{{C}_{2} S} + 866 \cdot {p}_{{C}_{3} A} + 420 \cdot {p}_{{C}_{4} AF} + 624 \cdot {p}_{{SO}_{3}} + 1186 \cdot {p}_{FreeCA} + 850 \cdot {p}_{MgO} \qquad Equation ( 8 )$$

$${H}_{u} = {H}_{cem} \cdot {p}_{cem} + 550 \cdot {p}_{slag} + 1800 \cdot {p}_{FA - CaO} \cdot {p}_{FA} + 330 \cdot {p}_{SF} \qquad Equation ( 9 )$$

$${E}_{a} = 41230 + 833000 ( ( {p}_{{C}_{3} A} + {p}_{{C}_{4} AF} ) \cdot {p}_{cem} \cdot {p}_{{SO}_{3}} \cdot {p}_{cem} ) - 347000 \cdot {p}_{{Na}_{2} Oeq} - 19.8 \cdot Blaine + 29600 \cdot {p}_{FA} \cdot {p}_{FA - CaO} + 16200 \cdot {p}_{slag} - 51600 \cdot {p}_{SF} - 3090000 \cdot WRRET - 345000 \cdot ACCL \qquad Equation ( 10 )$$

$$β = {e}^{( - 0.464 + \frac{3.41 \cdot {p}_{{C}_{3} A} \cdot cem - 0.846 \cdot SG}{{W}_{c m}} + 107 \cdot WRRET + 33.8 \cdot LRWR + 15.7 \cdot MRWR + 38.3 \cdot PCHRWR + 8.97 \cdot NHRWR )} \qquad Equation ( 11 )$$

$$τ = {e}^{( 2.92 + \frac{- 0.757 \cdot {p}_{{C}_{3} S} \cdot cem + 98.8 \cdot {p}_{{Na}_{2} O} \cdot cem + 1.44 \cdot slag + 4.12 \cdot FA \cdot {p}_{FA - CaO}}{{W}_{c m}} - 11.4 \cdot ACCL + 98.1 \cdot WRRET )} \qquad Equation ( 12 )$$

$${α}_{u} = \frac{1.031 \cdot {W}_{w} / {W}_{c m}}{0.194 + {W}_{w} / {W}_{c m}} + {e}^{( - 0.0885 + \frac{- 13.7 \cdot {p}_{{C}_{4} AF} \cdot cem - 283 \cdot {p}_{{Na}_{2} Oeq} \cdot cem - 9.9 \cdot FA \cdot {p}_{FA - CaO}}{{W}_{c m}} - 339 \cdot WRRET - 95.4 \cdot PCHRWR )} \qquad Equation ( 13 )$$

$${H}_{u} = ( ( 500 \cdot {p}_{{C}_{3} S} + 260 \cdot {p}_{{C}_{2} S} + 866 \cdot {p}_{{C}_{3} A} + 420 \cdot {p}_{{C}_{4} AF} + 624 \cdot {p}_{{SO}_{3}} + 1186 \cdot {p}_{FREECA} + 850 \cdot {p}_{MgO} ) \cdot cem + 550 \cdot SG + 330 \cdot SF + 1800 \cdot {p}_{FA - CaO} \cdot FA ) \cdot 1000 / {W}_{c m} \qquad Equation ( 14 )$$

$${E}_{a} = ( 41.23 + ( 833 ( ( {p}_{{C}_{3} A} + {p}_{{C}_{4} AF} ) \cdot {cem}^{2} \cdot \frac{{p}_{{SO}_{3}}}{{w}_{c m}} ) + 29.6 \cdot FA \cdot {p}_{FA - CaO} + 16.2 \cdot SG - 51.6 \cdot SF ) / {W}_{c m} - 347 \cdot {p}_{{Na}_{2} Oeq} - 0.0198 \cdot Blaine - 3090 \cdot WRRET - 345 \cdot ACCL ) \cdot 1000. \qquad Equation ( 15 )$$

Second Definition Module 1530 calculates an equivalent age of the concrete of the i^{th} time period, teᵢ (in hours), as given by Equation (17) which is the inventor modified form of Equation (16) for the Python based embodiment of the invention where Ea is the activation energy (J/mol), R is the universal gas constant (J/mol/K), Tᵣ is the reference temperature (° C.), tempᵢ is the concrete temperature (° C.), and dt is the time between iterations (in hours).

$${t}_{e} = {\int }_{0}^{t} {e}^{\frac{{E}_{a}}{R} ( \frac{1}{273 + {T}_{r}} - \frac{1}{273 + temp} )} dt \qquad Equation ( 16 )$$

$$t {e}_{i + 1} = t {e}_{i} + {e}^{\frac{{E}_{a}}{R} ( \frac{1}{273 + {T}_{r}} - \frac{1}{273 + tem {p}_{i}} )} \cdot dt \qquad Equation ( 17 )$$

Third Definition Module 1535 calculates the degree of hydration d equivalent age of the concrete i^{th} time period, αᵢ, as given by Equation (19) which is the inventor modified form of Equation (18) for the Python based embodiment of the invention where αᵤ is the ultimate degree of hydration, and t is the hydration time parameter (h), β is the hydration slope parameter.

$$α = {α}_{u} \cdot {e}^{- {( \frac{τ}{{t}_{e}} )}^{β}} \qquad Equation ( 18 )$$

$${α}_{i + 1} = {α}_{u} \cdot {e}^{- {( \frac{τ}{{f}_{{e}_{i + 1}}} )}^{β}} \qquad Equation ( 19 )$$

Fourth Definition Module 1540 calculates the specific heat of the concrete, c_{pconc}_{ᵢ₊₁}, as given by Equation (21) which is the inventor modified form of Equation (20) for the Python based embodiment of the invention for the i^{th} time period where cₜₒₜ is the cementitious materials average specific heat multiplied by respective weights

$$( \frac{J}{K \cdot {m}^{3}} )$$

as given by Equation (22), c_{dens} is the concrete density (kg/m³), αᵢ is the degree of hydration, W_{cm} is the weight of cementitious materials (kg/m³), tempᵢ is the concrete temperature (° C.), and c_{WAS} is the water, aggregates and sand average 13 specific heat multiplied by respective weights

$$( \frac{J}{K \cdot {m}^{3}} )$$

as given by Equation (23).

$${c}_{p c o n c} = \frac{1}{{ρ}_{c o n c}} ( {W}_{c} \cdot α \cdot {c}_{c e f} + {W}_{c} \cdot ( 1 - α ) \cdot {c}_{c} + {W}_{a} \cdot {c}_{a} + {W}_{s} \cdot {c}_{s} + {W}_{w} \cdot {c}_{w} ) \qquad Equation ( 20 )$$

$${c}_{c e f} = 8.4 \cdot {T}_{c} + 339$$

$${c}_{p c o n {c}_{i + 1}} = \frac{1}{{c}_{d e n s}} ( {c}_{tot} \cdot ( 1 - {α}_{i + 1} ) + {W}_{c m} \cdot {α}_{i + 1} \cdot ( 8.4 \cdot {temp}_{i} + 339 ) + {c}_{W A S} ) \qquad Equation ( 21 )$$

$${c}_{tot} = cem \cdot {c}_{c} + FA \cdot {c}_{F A} + SG \cdot {c}_{S G} + SF \cdot {c}_{S F} = 750 \cdot cem + 730 \cdot FA + 750 \cdot SF + 800 \cdot SG \qquad Equation ( 22 )$$

$${c}_{W A S} = {W}_{a} \cdot {c}_{a} + {W}_{s} \cdot {c}_{s} + {W}_{w} \cdot {c}_{w} = 840 \cdot {W}_{a} + 830 \cdot {W}_{s} + 4180 \cdot {W}_{w} \qquad Equation ( 23 )$$

Fifth Definition Module 1545 calculates, kᵢ, the concrete thermal conductivity

$$( \frac{W}{m \cdot K} )$$

via Equation (25) which is the inventor modified form of Equation (24) for the Python based embodiment of the invention for the i^{th} time period where k_{uc} is the ultimate concrete thermal conductivity

$$( \frac{W}{m \cdot K} )$$

and αᵢ is the degree of hydration.

$$k = {k}_{uc} \cdot ( 1.33 - 0.33 α ) \qquad Equation ( 24 )$$

$${k}_{i + 1} = {k}_{uc} \cdot ( 1.33 - 0.33 {α}_{i + 1} ) \qquad Equation ( 25 )$$

Sixth Definition Module 1150 calculates, α₍ⱼ₎₋₍ⱼ₊₁₎, the thermal transfer constant

$$( \frac{W}{K \cdot {m}^{2}} )$$

via Equation (27) which is the inventor modified form of Equation (26) for the Python based embodiment of the invention for the i^{th} time period for the concrete nodes and Equation (28) the inventor modified form of Equation (25) for the Python based embodiment of the invention for the i^{th} time period for the concrete nodes. In Equations (26) and (27) Dx is the distance between nodes (m), kⱼ is the thermal conductivity of concrete

$$( \frac{W}{m \cdot K} ) ,$$

fw_thick is the formwork thickness (m), and k_fw is the thermal conductivity of formwork

$$( \frac{W}{m \cdot K} ) .$$

$${a}_{( j ) - ( j + 1 )} = {[ \frac{{dx}_{j}}{{k}_{j}} + \frac{{dx}_{j + 1}}{{k}_{j + 1}} ]}^{- 1} Δ y \qquad Equation ( 26 )$$

$${a}_{( j ) - ( j + 1 )} = \frac{2}{Dx [ \frac{1}{{k}_{j}} + \frac{1}{{k}_{j + 1}} ]} \qquad Equation ( 27 )$$

$${a}_{( j ) - ( j + 1 )} = \frac{2}{[ \frac{fw\_thick}{k\_fw} + \frac{Dx}{{k}_{j + 1}} ]} \qquad Equation ( 28 )$$

Seventh Definition Module 1555 calculates, hi, the convection coefficient for a node exposed to air via Equation (30) which is the inventor modified form of Equation (29) for the Python based embodiment of the invention for the i^{th} time period where C is the heat flow constant, tempᵢ is the temperature in the exposed node (° C.), T_{α}_{ᵢ} is the ambient temperature (° C.), wᵢ is the wind speed (m/s), and R_{f} is the roughness multiplier.

$${h}_{i} = C \cdot 0.2782 \cdot ( {[ \frac{1}{{T}_{avg} + 17.8} ]}^{0.181} ) \cdot ( {\vert {T}_{s} - {T}_{a} \vert}^{0.266} ) \cdot \sqrt{1 + 2.8566 \cdot w} \cdot {R}_{f} \qquad Equation ( 29 )$$

$${h}_{i + 1} = C \cdot 0.2782 \cdot ( {[ \frac{1}{max ( \frac{{temp}_{i} + {T}_{{a}_{i + 1}}}{2} , - 16.8 ) + 17.8} ]}^{0.181} ) \cdot ( {\vert {temp}_{i} + {T}_{{a}_{i + 1}} \vert}^{0.266} ) \cdot \sqrt{1 + 2.8566 \cdot {w}_{i + 1}} \cdot {R}_{f} \qquad Equation ( 30 )$$

The inventors note that within the software Protection was added if an undefined or complex number is returned and that

$${[ \frac{1}{{T}_{avg} + 17.8} ]}^{0.181}$$

is set to never go higher than 1 to avoid errors. Table 2 presents roughness multipliers employed by the inventors for common formwork materials whilst Table 3 presents values of C, the heat flow constant.

TABLE 2
Roughness Multipliers for Common Framework Materials

| Material | R_{f} |
|---|---|
| Concrete | 1.52 |
| Steel | 1.00 |
| Wood | 1.00 |

TABLE 3
Heat Flow Constants

| | Vertical | Horizontal | Horizontal Top | |
|---|---|---|---|---|
| C values | “Ver” | Bottom“Hor_Bot” | “Hor_Top” | “None” |
| temp > Tₐ | 15.89 | 10.15 | 20.4 | 0 |
| temp < Tₐ | 15.89 | 20.4 | 10.15 | 0 |

It would be evident that other Configuration Modules may be employed in addition to those described. The data established within second Module Block 1500B, and data from modules within first Module Block 1500A is employed within third Module Block 1500C relating to establishing the temperature of each node within the simulated structure. Third Module Block 1500C comprises first to fourth Modelling Modules 1560 to 1575 respectively.

First Modelling Module 1560 calculates the energy flowing of a node of the modelled structure where in first Modelling Step 1560A the energy flowing into the node is calculated and then the energy flowing out of the node is calculated in second Modelling Step 1560B.

Accordingly within first Modelling Step 1560A the software executes Equation (32), which is the inventor modified form of Equation (31) for the Python based embodiment of the invention, for the i^{th} time period for Eᵢₙⱼ, the energy coming into a regular node j(w/m²), where tempⱼ is the temperature in the node j (° C.) and a₍ⱼ₎₋₍ⱼ₊₁₎ is the thermal transfer constant

$$( \frac{W}{K \cdot {m}^{2}} ) .$$

Similarly, within first Modelling Step 1560A the software executes Equation (34), which is the inventor modified form of Equation (33) for the Python based embodiment of the invention, for the i^{th} time period for Eᵢₙⱼ, the energy coming into a boundary node j(w/m²), where h is the convection coefficient and sol is the solar radiation (w/m²).

$${E}_{in} = {T}_{1} \cdot {a}_{1 - 2} + {T}_{3} \cdot {a}_{2 - 3} \qquad Equation ( 31 )$$

$${E}_{{in}_{j}} = {temp}_{j - 1} \cdot {a}_{( j - 1 ) - j} + {temp}_{j + 1} \cdot {a}_{j - ( j + 1 )} \qquad Equation ( 32 )$$

$${E}_{in} = {T}_{1} \cdot {a}_{1 - 2} + {T}_{∞} \cdot h \cdot Δ y \qquad Equation ( 33 )$$

$${E}_{{in}_{j}} = {temp}_{j + 1} \cdot {a}_{j - ( j + 1 )} + {T}_{a} \cdot h + sol \qquad Equation ( 34 )$$

Then, within the second Modelling Step 1560B the software executes Equation (36), which is the inventor modified form of Equation (35) for the Python based embodiment of the invention, for the i^{th} time period for Eₒᵤₜ_{ⱼ}, the energy coming out of a node j(w/m²), within the structure being modelled where tempⱼ is the temperature in the node j (° C.) and a₍ⱼ₎₋₍ⱼ₊₁₎ is the thermal transfer constant

$$( \frac{W}{K \cdot {m}^{2}} ) .$$

Similarly, within second Modelling Step 1560B the software executes Equation (38), which is the inventor modified form of Equation (37) for the Python based embodiment of the invention, for the i^{th} time period for Eₒᵤₜ_{ⱼ}, the energy coming out of a boundary node j(w/m²), where h is the convection coefficient, E_{c} is the concrete evaporation rate (kg/m²h) as given by Equation (39) (adapted form of estimated evaporation rates to prevent plastic shrinkage cracking in concrete) and hₗₐₜ=2500000+1859·tempⱼ (° C.).

$${E}_{out} = {T}_{2} \cdot {a}_{1 - 2} + {T}_{2} \cdot {a}_{2 - 3} \qquad Equation ( 35 )$$

$${E}_{{out}_{j}} = {temp}_{j} \cdot {a}_{( j - 1 ) - j} + {temp}_{j} \cdot {a}_{j - ( j + 1 )} \qquad Equation ( 36 )$$

$${E}_{out} = {T}_{2} \cdot {a}_{1 - 2} + {T}_{2} \cdot h \cdot Δ y \qquad Equation ( 37 )$$

$${E}_{{out}_{j}} = {temp}_{j} \cdot {a}_{j - ( j + 1 )} + {temp}_{j} \cdot h + {h}_{lat} \cdot {E}_{c} \qquad Equation ( 38 )$$

$${E}_{c} = 21.2234 \cdot ( {( {temp}_{j} + \frac{160}{9} )}^{2.5} - r \cdot {( {T}_{a} + \frac{160}{9} )}^{2.5} ) \cdot ( 0.2485 \cdot w + 1 ) * \frac{{10}^{- 6}}{3600} \qquad Equation ( 39 )$$

Within Second Modelling Module 1565 the software calculates, the energy generated in the node, E_{gen}_{ᵢ} (w/m²), where Qᵢ is the heat generated (w/m²) and Dx is the distance between nodes (m) within the model. The heat generated, Qᵢ, is calculated using Equation (42) which is the inventor modified form of Equation (41) for the Python based embodiment of the invention, for the i^{th} time period the heat generated, Qᵢ (w/m²), where β is the hydration slope parameter, τ is the hydration time parameter (h), αᵤ is the ultimate degree of hydration, Hᵤ is the total amount of heat generated at 100% hydration (J/kg), E_{α} is the activation energy (J/mol),

W_{cm} is the weight of cementitious materials (kg/m³), teᵢ is the equivalent age (h), R is the universal gas constant

$$( 8.314 \frac{J}{mol \cdot K} ) ,$$

Tᵣ is the reference temperature (which is typically 20° C. or 23° C. for US based locations), and tempᵢ is the temperature in the node (° C.)

$${E}_{{gen}_{i}} = {Q}_{i} \cdot Dx \qquad Equation ( 40 )$$

$$Q = {H}_{u} \cdot {C}_{c} \cdot {( \frac{τ}{{t}_{e}} )}^{β} \cdot ( \frac{β}{{t}_{e}} ) \cdot {α}_{u} \cdot {e}^{- {( \frac{τ}{{t}_{e}} )}^{β}} \cdot {e}^{( \frac{{E}_{a}}{R} \cdot ( \frac{1}{273 + {T}_{r}} - \frac{1}{273 + {T}_{2}} ) )} \cdot \frac{1}{3600} \qquad Equation ( 41 )$$

$${Q}_{i} = {H}_{u} \cdot W\_cm \cdot {( \frac{τ}{{t}_{{e}_{i}}} )}^{β} \cdot ( \frac{β}{{t}_{{e}_{i}}} ) \cdot {α}_{u} \cdot {e}^{- {( \frac{τ}{{t}_{{e}_{i}}} )}^{β}} \cdot {e}^{( \frac{{E}_{a}}{R} ( \frac{1}{273 + {T}_{r}} - \frac{1}{273 + {temp}_{i}} ) )} \cdot \frac{1}{3600} \qquad Equation ( 42 )$$

In third Modelling Module 1570 the software calculates ΔE, the energy variation in a node of the model, as defined in Equation (43), where Eᵢₙ is the energy coming in the node (w/m²), Eₒᵤₜ is the energy coming out of the node (w/m²), and E_{gen} is the energy generated in the node W This is calculated using Equation (45) which is the inventor modified form of Equation (44) for the Python based embodiment of the invention for the i^{th} time period where tempᵢ is the temperature in the node (° C.), ΔEᵢ is the energy variation in the node, c_{dens} is the material density (kg/m³), cₚ_{ᵢ} is the material specific heat,

$$( \frac{J}{kg \cdot K} ) ,$$

Dx is the distance between nodes (m), and dt is the time between iterations (h).

$$Δ E = {E}_{in} - {E}_{out} + {E}_{gen} \qquad Equation ( 43 )$$

$$Δ E = \frac{ρ \cdot {c}_{p} \cdot Δ x \cdot Δ y \cdot ( {T}^{+ 1} - T )}{Δ t} \qquad Equation ( 44 )$$

$${temp}_{i + 1} = \frac{Δ {E}_{i} \cdot dt}{{c}_{dens} \cdot {c}_{{p}_{i}} \cdot Dx} \cdot 3600 + {temp}_{i} \qquad Equation ( 45 )$$

Accordingly, by performing the calculations for each node for each subsequent time interval the software model according to embodiments of the invention calculates the net flow of energy within each node and accordingly establishes the temperature of the node(s) over time. As noted above the software solution established by the inventors models the thermal evolution of a concrete structure to within a few degrees. The maximum deviation between simulated temperature and measured temperature for the structures implemented was 2.8° C. (5 F).

### Ticket—Certificate Data Extraction (Unstructured Data Source to Structured Data Conversion)

Within FIGS. 6 to 10 there are described and depicted software based systems related to the specification of, manufacture of, quality control of, and life cycle monitoring of construction materials such as concrete. A construction project may employ a single batch or multiple batches of a construction material where the raw materials employed within the construction material as well as the batch(es) of construction material each have data associated with them which is provided through unstructured sources such as concrete batching tickets (certificates), delivery tickets, hand-written reports of concrete historical data, mill certificates of cementitious materials performance, aggregate particle size distribution reports, printed documents showing concrete mixture proportions, delivery tickets showing the as-delivered concrete properties (e.g. flowability of concrete) as well as concrete testing results from various producers. However, the software based systems related to the specification of, manufacture of, quality control of, and life-cycle monitoring of construction materials require that the data from these unstructured sources is organized and labelled for entry into the one or more databases associated with the software based systems.

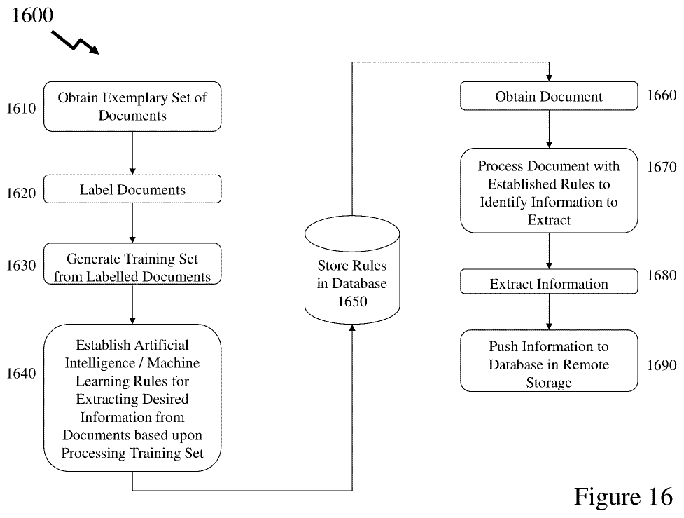

Accordingly, the inventors have established a process to acquire, extract, label (if required) and store the data within these one or more databases associated with the software based systems. An exemplary process Flow 1600 for this process is depicted in FIG. 16 comprising first to ninth steps 1610 to 1690 respectively, these comprising:
First step 1610 wherein an exemplary set of documents are obtained;
Second step 1620 wherein each document of the exemplary set of documents is labelled to identify those regions containing content which should be extracted;
Third step 1630 wherein a training set of documents are generated from the labelled set of documents;
Fourth step 1640 wherein the training set of documents are processed by a system employing machine learning (ML) based and/or artificial intelligence (AI) based rule generators which generate a set of rules for extracting content from unstructured documents in dependence upon the labelled content in each document of the training set of documents;
Fifth strep 1650 wherein the rules are stored within a database;
Sixth step 1660 wherein a document is obtained relating to an aspect of the generation, transport, deployment, etc. of a construction material;
Seventh step 1670 wherein the document is processed, e.g. via image processing, in conjunction with the established rules stored in the database to identify whether regions having content to be extracted are present in the document;
Eighth step 1680 wherein content within the identified regions having content to be extracted is extracted, for example by other image processing, character recognition etc.; and
Ninth step 1690 wherein the information comprising the content within the identified regions having content to be extracted is stored within remote storage, e.g. one or more other databases accessible to the software based systems related to the specification of, manufacture of, quality control of, and life cycle monitoring of construction materials.

It would be evident that if in seventh step 1670 a determination is made that there are no regions having content to be extracted present within the document the process for that document may terminate and the process re-starts when another document is obtained at sixth step 1660.

Within embodiments of the invention the processes are described as employing character recognition and/or optical imaging processes. It would be evident that such processes can be employed upon scanned/imaged printed documents or upon electronically generated printed versions of documents. Accordingly, the source documents may be printouts provided with a delivery of a batch of material or a spreadsheet or other item of electronic content which is processed. Accordingly, the conversion of electronic documents to a format compatible with character recognition and/or optical imaging processes removes any requirements for the software extracting content to ascertain relationships between elements within the electronic document by processing formatting and layout information within the document. Working with an electronic “printed” version removes these issues as the printed version has the formatting established and rendered within the printed version.

Within an embodiment of the invention the labelling of documents is performed manually. Within other embodiments of the invention the labelling of the documents may employ one or more rules already established within the database wherein a software application employed at this stage identifies other regions likely to contain information which should be labelled wherein another region likely to contain information may, for example, be automatically labelled if a threshold is exceeded or labelled if a user subsequently presented with the document and an indication of a region likely to contain information defines the region as one to be labelled. Accordingly, the process depicted in FIG. 16 through first to fifth steps 1650 may be an iterative learning AI or ML based process for identifying regions of documents to be labelled for extraction of content.

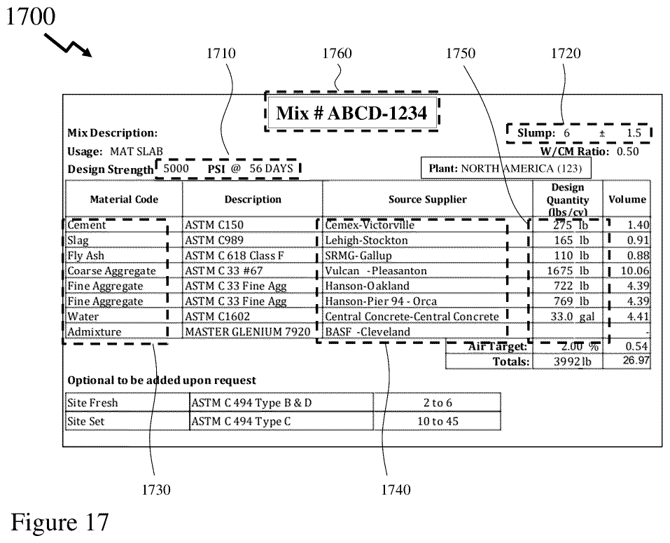

Examples of the collected unstructured data identified within documents relating to the specification of, manufacture of, quality control of, and life-cycle monitoring of construction materials are depicted within FIGS. 17 to 19 respectively. Referring to FIG. 17 there is depicted a “Mix Certificate” 1700 wherein first to sixth Labelled Regions 1710 to 1760 are identified defining:
First Labelled Region 1710 identifying the design strength of the construction material;
Second Labelled Region 1720 identifying a measured property of the construction material, in this instance “Slump”, together with the result for this batch of material;
Third Labelled Region 1730 identifying materials within the mix of the construction material, e.g. cement, slag, fly ash etc.;
Fourth Labelled Region 1740 identifying the source suppliers of each material;
Fifth Labelled Region 1750 identifying the quantities of the each material;
Sixth Labelled Region 1760 representing a unique identifier of the batch of the construction material.

Within embodiments of the invention the labelling may associate labelled regions rather than identifying them as individual regions. Accordingly, first to sixth Labelled Regions 1710 to 1760 may be associated or a subset of first to sixth Labelled Regions 1710 to 1760 may be associated.

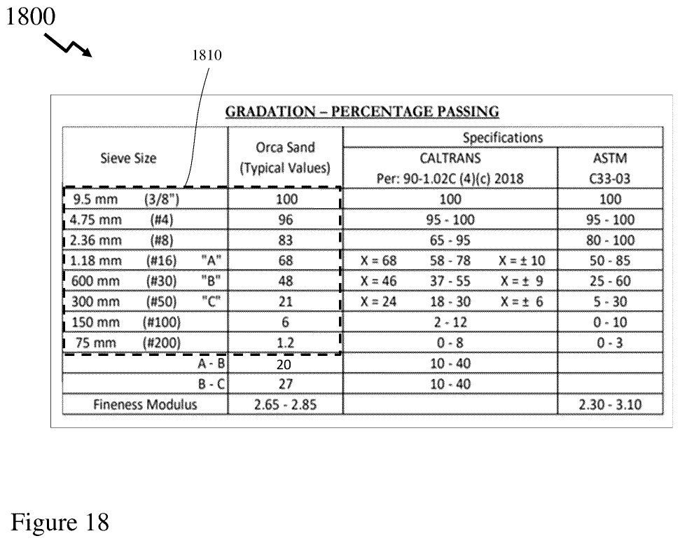

Referring to FIG. 18 there is depicted a “Gradation Certificate” 1800 for a batch of material (e.g. sand, aggregate etc.) employed by a construction material producer. Labelled Region 1810 within “Gradation Certificate” 1800 defines different sieve dimensions and the measured results for the batch of material.

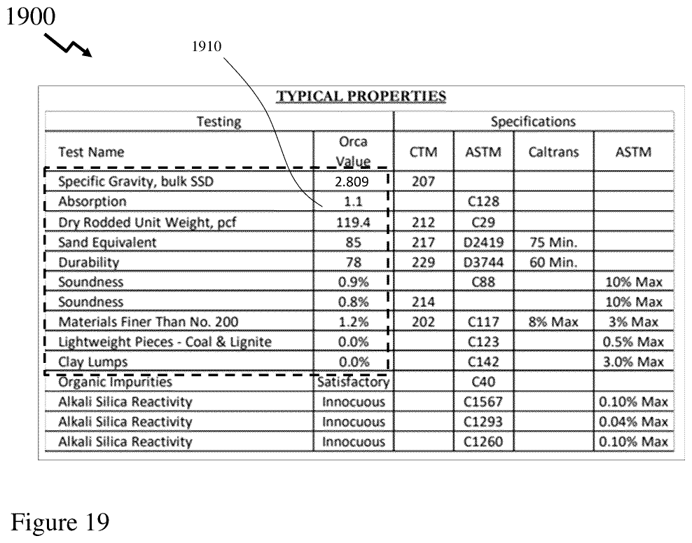

Now referring to FIG. 19 there is depicted a “Properties Certificate” 1900 for a batch of material employed by a construction material producer. Labelled Region 1910 within “Properties Certificate” 1800 defines different tests performed on the batch of material and the measured results for each test upon the batch of material.

It would be evident that even with a single construction project multiple suppliers of a single material may be employed even before considering the multiple materials etc. As each supplier will have established their own documentation then an enterprise or organization responsible for the construction project is faced with a massive data management/acquisition process if they were to do this pre-emptively prior to any issue arising during the lifetime of the construction project or at a point in time that an issue arises. For large construction companies these issues are multiplied by the number of projects they manage either within a single location, state, region, country or multi-nationally where issues over language, different testing standards, etc. further compound the data management/acquisition process.

Accordingly, the ML/AI processes and algorithms established by the inventors are essential for a data-management platform(s) for specification of, manufacture of, quality control of, and life-cycle monitoring of one or more construction materials in order to successfully scale automated data collection across a large number of unstructured sources of data, usually printed or supplied in an electronic format such as a portable document format (PDF). The algorithm development work included development of convolutional neural networks and residual neural networks capable of ingesting data from these documents through character recognition. These algorithms are able to view these documents, infer the data within the documents, organize it in the format required for algorithm training and subsequently feed the collected data to the algorithm for training purposes.

Accordingly, clients to a provider of software application(s) and/or data-management platform(s) for specification of, manufacture of, quality control of, and life-cycle monitoring of one or more construction materials are able to sign-up, upload their historical data to the platform in a simple manner without the need for any manual processing etc. by the client, wherein the data management platform ingests the historical documents, generates the rules, and then automatically processes new data as it is uploaded by the client directly or by suppliers to the client associated with the client within the software application/data-management platform(s)/database(s) etc.

### Cement “DNA”

Different standards and specifications require cement producers to supply concrete producers with what are referred to as a “mill certificate” (formally a Mill Test Certificate or Mill Test Report). This identifies the chemical and physical characteristics of a batch of cement including, for example, the chemical composition of the cement, its fineness, its autoclave expansion, its setting time and its compressive strength according to the applicable standards.

A factor hindering concrete producers from utilizing this data to optimize their concrete mixtures according to the quality of the received cement is that such mill certificates are typically an average of large production runs (with varying quality) and therefore are not representative of the specific cement batch received by the producer. For example, some cement producers generate mill certs representing over 200,000 metric tons of cement.

Accordingly, the inventors have established a method to determine the strength of the cement by measuring the impedance and permittivity characteristics of the cement from which a porosity of the cement can be determined. The inventors having previously established this methodology for concrete. However, in contrast to the concrete methodology as cement testing is performed within a non-air-entrained condition then the method established by the inventors does not require an additional measurement of the air content. Accordingly, the measurements do not require a measurement of the speed of sound of the concrete (such as performed by Giatec Scientific's SmartRock™ sensor (SmartRock™ 4) which performs a measurement of porosity of concrete implemented by the inventors) to determine its air content. Accordingly, the measurements may be performed by deploying sensors within the cement which subsequently are either removed through a screening process or left to be embedded with the final concrete mixture.

### Aggregate Gradation

Within FIGS. 6 to 10 there are described and depicted software based systems related to the specification of, manufacture of, quality control of, and life cycle monitoring of construction materials such as concrete. Such construction materials may employ particulate matter, such as aggregate in the instance of concrete. Accordingly, the inventors have established a method allowing for the continuous or periodic monitoring of the particulate gradation.

Accordingly, the inventive method established by the inventors exploits a software process to establish a model based upon watershed transforms to perform particle segmentation from images of particulates (e.g. aggregates) of varying sizes. This model is then used in conjunction with a convolutional neural network, or another form of AI/ML system, to classify aggregates into those of interest isolating artifacts in acquired images of the aggregates from those caused by image noise or distortion. Accordingly, the model/process then analyses the identified objects of interest in order to establish parameters relating to the particulates. For example, for aggregates these parameters may include, but not be limited to, angularity, shape factor, particle-size distribution, aspect ratio and Ferret diameter.

Accordingly, the knowledge of the particulate properties employed in forming a construction material can then be employed within software based systems related to the specification of, manufacture of, quality control of, and life-cycle monitoring of construction materials. For example, the data may be employed to optimize a packing density of a construction material mixture. Alternatively, the data may be employed in conjunction with other data relating to mixture rheology of a construction material mixture employing the particulates to which the data relates in order to optimize one or more parameters of the mixture which may include, but not be limited to, cost, flowability, compressive strength, tensile strength, cure properties, and lifetime.

An aggregate may be viewed as a particulate mixture comprising a plurality of aggregate particles.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps, and means described above may be done in various ways. For example, these techniques, blocks, steps, and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above and/or a combination thereof.

Also, it is noted that the embodiments may be described as a process which is depicted as a flowchart, a flow diagram, a data flow diagram, a structure diagram, or a block diagram. Although a flowchart may describe the operations as a sequential process, many of the operations can be performed in parallel or concurrently. In addition, the order of the operations may be rearranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages and/or any combination thereof. When implemented in software, firmware, middleware, scripting language and/or microcode, the program code or code segments to perform the necessary tasks may be stored in a machine readable medium, such as a storage medium. A code segment or machine-executable instruction may represent a procedure, a function, a subprogram, a program, a routine, a subroutine, a module, a software package, a script, a class, or any combination of instructions, data structures and/or program statements. A code segment may be coupled to another code segment or a hardware circuit by passing and/or receiving information, data, arguments, parameters and/or memory content. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor and may vary in implementation where the memory is employed in storing software codes for subsequent execution to that when the memory is employed in executing the software codes. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more devices for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels and/or various other mediums capable of storing, containing, or carrying instruction(s) and/or data.

The methodologies described herein are, in one or more embodiments, performable by a machine which includes one or more processors that accept code segments containing instructions. For any of the methods described herein, when the instructions are executed by the machine, the machine performs the method. Any machine capable of executing a set of instructions (sequential or otherwise) that specify actions to be taken by that machine are included. Thus, a typical machine may be exemplified by a typical processing system that includes one or more processors. Each processor may include one or more of a CPU, a graphics-processing unit, and a programmable DSP unit. The processing system further may include a memory subsystem including main RAM and/or a static RAM, and/or ROM. A bus subsystem may be included for communicating between the components. If the processing system requires a display, such a display may be included, e.g., a liquid crystal display (LCD). If manual data entry is required, the processing system also includes an input device such as one or more of an alphanumeric input unit such as a keyboard, a pointing control device such as a mouse, and so forth.

The memory includes machine-readable code segments (e.g., software or software code) including instructions for performing, when executed by the processing system, one of more of the methods described herein. The software may reside entirely in the memory, or may also reside, completely or at least partially, within the RAM and/or within the processor during execution thereof by the computer system. Thus, the memory and the processor also constitute a system comprising machine-readable code.

In alternative embodiments, the machine operates as a standalone device or may be connected, e.g., networked to other machines, in a networked deployment, the machine may operate in the capacity of a server or a client machine in server-client network environment, or as a peer machine in a peer-to-peer or distributed network environment. The machine may be, for example, a computer, a server, a cluster of servers, a cluster of computers, a web appliance, a distributed computing environment, a cloud computing environment, or any machine capable of executing a set of instructions (sequential or otherwise) that specify actions to be taken by that machine. The term “machine” may also be taken to include any collection of machines that individually or jointly execute a set (or multiple sets) of instructions to perform any one or more of the methodologies discussed herein.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the scope of the present invention.

1. The method according to claim 13, wherein
the characteristic of the construction material is a yield stress of the load of construction material;
the process comprises:
providing a drum containing a load of the construction material;
providing at least one of an accelerometer and a gyroscope attached to the drum;
determining a torque of an engine rotating the drum at a plurality of rates of revolution;
determining in dependence upon the torque of the engine at the plurality of different revolutions per minute (RPM), parameters of the drum, and a weight of a construction material within the drum the yield stress of the construction material; and
the yield stress of the construction material is established by extrapolating the torque of the engine at the plurality of rates of revolution to a torque required at zero (0) RPM.

2. The method according to claim 13, wherein
the characteristic of the construction material is a slump of the construction material and the construction material is concrete;
the process comprises:
providing a drum containing a load of concrete;
providing a hydraulic pressure sensor measuring pressure of a hydraulic system associated with the drum;
providing a sensor to monitor an angular velocity of the drum;
monitoring the hydraulic pressure of the hydraulic system over a range of angular velocities with the drum empty;
monitoring the hydraulic pressure of the hydraulic system over a range of angular velocities with a load of concrete within the drum;
determining in dependence upon the difference in hydraulic pressures between the drum empty and loaded with concrete over the range of angular velocities and the range of angular velocities a shear rate and a shear stress applied to the concrete;
determining a yield stress of the concrete from the shear rate and a shear stress; and
determining in dependence upon the yield stress of the concrete the slump of the concrete.

3. The method according to claim 13, wherein
the characteristic of the construction material is a size of a concrete load;
the process comprises:
providing a drum containing the load of concrete;
providing a hydraulic pressure sensor measuring pressure of a hydraulic system associated with the drum;
providing a sensor to monitor an angular velocity of the drum;
monitoring the hydraulic pressure of the hydraulic system over a range of angular velocities with the drum empty;
monitoring the hydraulic pressure of the hydraulic system over a range of angular velocities with a load of concrete within the drum;
determining in dependence upon the hydraulic pressures over the range of angular velocities when the drum is empty and the range of angular velocities a system efficiency value; and
determining in dependence upon the hydraulic pressures over the range of angular velocities when the drum is loaded, the range of angular velocities and the system efficiency value the size of the concrete load.

4. The method according to claim 13, wherein
the characteristic of the construction material is at least one of a flowability measurement or a slump measurement of the construction material;
the process comprises:
establishing a first pressure measurement from a first diaphragm based pressure sensor in contact with a column of a construction material at a first position with respect to the column of the construction material;
establishing a second pressure measurement from a second diaphragm based pressure sensor in contact with a column of a construction material at a second position with respect to the column of the construction material;
establishing a pressure differential in dependence upon the first pressure measurement and the second pressure measurement;
establishing a vertical separation between the first diaphragm based pressure sensor and the second diaphragm based pressure sensor; and
establishing the at least one of the flowability measurement or the slump measurement of the construction material.

5. The method according to claim 4, wherein
the first diaphragm based pressure sensor and the second diaphragm based pressure sensor form part of a probe;
the probe further comprises an inclinometer; and
the vertical separation is established in dependence upon a measurement of the inclinometer.

6. The method according to claim 13, wherein
the characteristic of the construction material is a measure of temperature increase of a body of liquid concrete at a plurality of points and a plurality of time;
the process comprises:
establishing a model comprising a formwork and a body of liquid concrete poured into the formwork;
executing a simulation process upon the model over a period of time to establish the measure of temperature increase of the body of liquid concrete at the plurality of points and the plurality of time; and
the simulation process has been correlated to a plurality of physical structures which have been modelled with the simulation process.

7. A method comprising:
establishing an initial set of documents;
labelling each document of the set of documents to identify within that document of the set of documents a plurality of regions within the document of the set of documents containing content of interest;
establishing a training set of documents from the labelled set of documents;
employing at least one of a machine learning (ML) based rule generator and an artificial intelligence (AI) based rule generator upon the training set of documents to generate a set of rules for extracting content in dependence upon the labelled content in each document of the training set of documents; and
storing the rules within the database.

8. The method according to claim 7, wherein
the set of documents are from a plurality of sources; and
the set of documents are unstructured.

9. The method according to claim 7, further comprising
obtaining a document relating to an aspect of the generation, transport, or deployment of a construction material;
processing the document with one or more image processing processes in conjunction with the established rules stored in the database to identify whether any regions of the document having content to be extracted are present in the document;
upon a positive determination that a region of the document is present that has content to be extracted extracting the content with one or more other image processing processes;
storing the extracted content within a database; wherein
the database is accessible to a software based system related to at least one of the specification of, the manufacture of, a quality control of, and a life cycle monitoring of a construction material.

10. The method according to claim 13, wherein
the characteristic of the construction material is at least one of a porosity of a cement, a time of set of the at least one of a cement paste and a mortar and a compressive strength of the at least one of the cement and the mortar;
the process comprises:
performing an electrical impedance measurement of at least one of the cement and the mortar with a sensor embedded within performing a permittivity measurement of the at least one of the cement and the mortar with the sensor; and
determining in dependence upon the electrical impedance measurement and the permittivity measurement the at least one of the porosity of the cement, the time of set of the at least one of the cement paste and the mortar and the compressive strength of the at least one of the cement and the mortar.

11. A method comprising:
establishing a model in dependence upon applying watershed transforms to images of particulates of varying sizes to perform particle segmentation;
employing a machine learning process or artificial intelligence based process to classify images of a particulate mixture to identify a plurality of aggregate particles within the particulate mixture by isolating the plurality of aggregate particles from artifacts within acquired images of the particulate mixture caused by at least one of image noise and distortion;
establishing one or more parameters of the plurality of aggregate particles in dependence of the model.

12. The method according to claim 11, wherein
one or more parameters of the plurality of aggregate particles are selected from the group comprising angularity, shape factor, particle-size distribution, aspect ratio and Ferret diameter.

13. A method comprising:
establishing a characteristic of a construction material via a process.
